(19) United States
(12) Patent Application Publication
(10) Pub. No.: US 2015/0057316 A1
(43) Pub. Date: Feb. 26, 2015

(54) NOVEL SULFONATE-BASED TRIMEBUTINE SALTS

(71) Applicant: GICARE PHARMA INC., Montreal Quest (CA)

(72) Inventors: Jean-Francois Meunier, Montreal (CA); Cheuk Kun Lau, Ile Bizard (CA); Daniel Guay, Lachine (CA); Gregory Bydlinski, Kirkland (CA); Nadejda Spassova, Montreal (CA); Louis-David Cantin, Beaconsfield (CA); Maxime Ranger, Montreal (CA)

(21) Appl. No.: 14/383,999

(22) PCT Filed: Mar. 11, 2013
(86) PCT No.: PCT/CA2013/050178

§ 371 Date: Sep. 9, 2014

Related U.S. Application Data
(60) Provisional application No. 61/609,543, filed on Mar. 12, 2012.

(51) Int. Cl. C07C 229/38; C07C 309/30; C07D 277/36; C07C 309/39; C07D 213/71; C07C 309/42; C07C 309/08; C07C 309/58; C07C 309/04; C07C 327/48; C07C 309/46

(52) U.S. Cl. CPC C07C 229/38; C07C 327/48; C07C 309/30; C07D 277/36; C07C 309/39; C07C 309/46; C07C 309/42; C07C 309/08; C07C 309/58; C07C 309/04; C07D 213/71
USPC 514/347; 560/73; 514/534; 548/186; 514/369; 558/412; 514/520; 546/294

(57) ABSTRACT

The present description relates to compounds of Formula I (A⁺ X⁻), a diastereoisomer, an enantiomer, or a mixture thereof, pharmaceutical composition comprising same and uses thereof for gastrointestinal endoscopic and medical imaging applications, and for the treatment of visceral pain: Where R₁ and R₂ are as defined herein

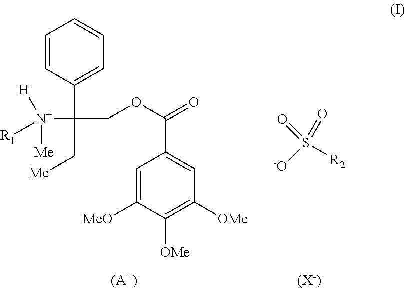

### FIELD

The present description relates to novel salts of trimebutine and N-monodesmethyl trimebutine, and their corresponding stereoisomers, and their analgesic properties in order to manage and relieve visceral pain. Such salts aims to manage and reduce visceral pain experienced by patients who either undergo colonoscopy, sigmoidoscopy, proctosigmoidoscopy, virtual colonoscopy or barium enema, or suffer from a gastrointestinal condition including, but not limited to, ulcerative colitis, internal and external hemorrhoids, radiation proctitis, all forms of irritable bowel syndrome (IBS) and other functional disturbances of gastrointestinal motility.

### BACKGROUND

Trimebutine[3,4,5-trimethoxybenzoic acid 2-(dimethylamino)-2-phenylbutylester] under its maleate salt form was first approved in France in 1969 as a spasmolytic drug and has been marketed in several countries since then for the treatment of functional bowel disorders, including IBS.

Trimebutine maleate has also been reported to be active against rectal hyperalgesia induced by local inflammation and stress in rats [Lacheze et al. (1998) J. Pharm: Pharmacol. 50: 921-928 “Influence of Trimebutine on Inflammation-and Stress-induced Hyperalgesia to Rectal Distension in Rats”].

Trimebutine was first described as an opioid agonist with micromolar affinities to mu- and kappa-opioid receptors [Roman et al. (1987) J. Pharm. Pharmacol. 39:404-407 “Interactions of trimebutine with guinea pig opioid receptors”], and some of its action is also attributed to the release of gastrointestinal peptides such as motilin and the modulation of the release of other peptides, including vasoactive intestinal peptide, gastrin and glucagon. Moreover, trimebutine accelerates gastric emptying, induces premature phase III of the migrating motor complex in the intestine and modulates the contractile activity of the colon [Chaussade S et al. (1987) Eur J Clin Pharmacol. 32(6):615-8. “Induction of phase III of the migrating motor complex in human small intestine by trimebutine”].

Later on, Roman et al. (1999) [J. Pharmacol. Exp. Ther. 289: 1391-1397 “Pharmacological Properties of Trimebutine and N-Monodesmethyltrimebutine” demonstrated that trimebutine and its active metabolite, N-desmethyl trimebutine, feature blocking activity on sodium channels providing significant local anaesthetic activity. In that study, N-desmethyl trimebutine featured higher activity than trimebutine on the blockage of sodium channels, and the (S)—N-desmethyltrimebutine seems to be the most active stereoisomer. However, although stereospecificity of drug action on sodium channel has been observed, other stereoisomers, including those of trimebutine, also showed significant activity.

Further studies demonstrated that the effects of trimebutine on the gastrointestinal tract were mediated by a calcium antagonist-like action, inhibiting the influx of extracellular Ca²⁺ in the smooth muscles cells [Shimada et al. (1990) J. Gastroenterol. 25:175-179 “Trimebutine maleate has inhibitory effects on the voltage-dependent Ca²⁺ inward current and other membrane currents in intestinal smooth muscle cells”; Nagasaki et al. (1991) Eur. J. Pharmacol. 195: 317-321 “Effects of trimebutine on cytosolic Ca2+ and force transitions in intestinal smooth muscle”]. Trimebutine was also associated with the inhibition effect of potassium current through membrane depolarization of the gastrointestinal smooth muscle cells at the resting conditions to induce contractions [Nagasaki et al. (1993) Eur. J. Pharmacol. 235: 197-203 “Effect of trimebutine on K+ current in rabbit ileal smooth muscle cells”]. V. Sinniger et al. (2005) [Life Sciences 77: 2927-2941 “Effect of nor-trimebutine on neuronal activation induced by a noxious stimulus or an acute colonic inflammation in the rat”] reported that N-desmethyl trimebutine decreased Fos expression in the thoraco-lumbar (peritoneal irritation) and lumbo-sacral (colonic inflammation) in different laminae of the spinal cord.

Furthermore, trimebutine has also been shown to decrease reflexes induced by distension of the gut lumen in animals and consequently to modulate visceral sensitivity [Delvaux M et al. (1997) J. Int. Med. Res. 25(5):225-46. “Trimebutine: mechanism of action, effects on gastrointestinal function and clinical results”].

Several clinical studies have demonstrated that trimebutine is efficacious to relieve abdominal pain as, for instance, reported in Ghidini et al. (1986) [Curr Ther Res 39: 541-548 “Single drug treatment for irritable colon: Rociverine versus trimebutine maleate”]. Trimebutine was proven to be effective in the treatment of both acute and chronic abdominal pain in patients with functional bowel disorders, especially IBS, at doses ranging from 300 to 600 mg/day. It is also effective in children presenting with abdominal pain.

It has been shown that hydrogen sulfide (H₂S) releasing agents exhibit analgesic activity in models of visceral pain [Distrutti et al. (2005) J. Pharmacol. Exp. Ther. 316: 325-335 “Evidence that hydrogen sulfide exerts antinociceptive effects in the gastrointestinal tract by activating KATP channels”; Distrutti et al. (2010) Molecular Pain 6:36 “Hydrogen sulphide induces μ opioid receptor-dependent analgesia in a rodent model of visceral pain. PCT/CA2007/001008, “Salts of Trimebutine and N-Desmethyl Trimebutine” relates to the preparation of hydrogen-sulfide trimebutine salts for the treatment of gastrointestinal disorders, such as IBS. Distrutti et al. (2009) [Pharm. Res. 59: 319-329 “A nitroarginine derivative of trimebutine (NO₂-Arg-Trim) attenuates pain induced by colorectal distension in conscious rats”] reported the preparation of a nitric oxide-releasing trimebutine salt, using L-nitroarginine as counter-ion, for the attenuation of pain experienced by rats under colorectal distension.

There is a renewed interest to use opioid agonists for the treatment of gastrointestinal disorders associated with visceral pain. Trimebutine remains a molecule of interest and its efficient analgesic effect has been shown when combined with a gaseous mediator-releasing moiety. Nevertheless, there are still unmet medical needs, in order to treat conditions such as irritable bowel disorders (IBS). More stable trimebutine salts should be developed for a pharmaceutical use. It is therefore be highly desirable to provide patients with improved trimebutine salt derivatives, which are thermodynamically stable in their solid-state and when administered to human beings, are able to help manage and reduce the visceral pain experienced with gastrointestinal disorders such as IBS or during lower gastrointestinal endoscopy such as colonoscopy.

### DESCRIPTION

The present description relates to trimebutine and N-desmethyltrimebutine salt compounds.

The present description relates to a salt compound of the general formula, A⁺X⁻, where the cation A⁺ is the protonated form of trimebutine, N-desmethyltrimebutine or one of their stereoisomers, and the anion X⁻ is a sulfonate derivative.

One aspect of the present description is a compound of Formula I (A⁺ X⁻), a diastereoismer, an enantiomer, or a mixture thereof:

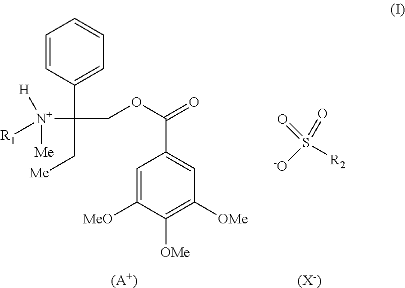

wherein:
R₁ is hydrogen or methyl;
R₂ is substituted or unsubstituted aryl, substituted or unsubstituted heteroaryl, substituted or unsubstituted alkyl, substituted or unsubstituted alkenyl or substituted or unsubstituted alkynyl.

In one aspect, the present description relates to pharmaceutical compositions comprising at least one salt compound as defined herein and a pharmaceutically acceptable excipient or carrier.

In a further aspect the present description relates to a method for reducing visceral pain of a patient, comprising the administration of a visceral pain relieving amount of a pharmaceutical composition as defined herein or of at least one salt compound as defined herein to a patient in need thereof.

In a further aspect, the present description relates to the use of a compound as defined herein in the preparation of a medicament for reducing visceral pain experienced by a patient.

In a further aspect, the present description relates to the use of a compound or composition as defined herein for reducing visceral pain experienced by a patient.

It has been shown that acetic acid (pKa ˜4.8), benzoic acid (pKa ˜4.2) and 4-thiocarbamoylbenzoic acid (pKa ˜3.3) respectively resulted in an unstable salt of trimebutine on 1:1 molar ratio, in the solid state. Proton exchange was observed in methanol when such acidic molecules were added to trimebutine and salt formation was observed upon rapid methanol evaporation. However, the salts tended to dissociate with heat and/or time and were converted back into separate trimebutine and acidic molecule. In addition to the unusual low pKa of trimebutine of ˜6.25 [Nagasaki et al. (1993) Br. J. Pharmacol. 110: 399-403 “Effect of trimebutine on voltage-activated calcium current in rabbit ileal smooth muscle cells”], the steric hindrance around the amine group of trimebutine also renders the salt formation difficult.

The trimebutine salts of PCT/CA2007/001008 and Distrutti et al. (2009) [Pharm. Res. 59: 319-329] are made from an alkylcarboxylic or an arylcarboxylic acid moiety. The present inventors have found that such salts are not thermodynamically stable in their solid state and may dissociate over time with external stimuli (e.g. heat, moisture). An acceptable pharmaceutical, solid dosage form requires using a drug salt which is very stable over time, ideally thermodynamically stable, in order to not alter the physicochemical and pharmacokinetic properties of the said drug.

In one aspect, certain salts of the present description showed a remarkable stability over time and over temperature, in addition to being well absorbed.

In a further aspect, certain salts of the present description have improved analgesic effects for the management and reduction of visceral pain experienced by patients during an endoscopic procedure or suffering from a gastrointestinal disorder induced pain.

The present description relates to the preparation of novel trimebutine salts in which the counter-ion is a sulfonate-based derivative. Sulfonate-based derivatives are the anion form of their sulfonic acid form. Alkylsulfonic acid, heteroarylsulfonic acid and arylsulfonic acid derivatives generally have pKa below 1. Moreover, this pKa decreases when electron-withdrawing groups are added to the aromatic ring of the arylsulfonate derivative. The difference in pKa between trimebutine and such sulfonic acid derivatives is large enough (difference in pKa exceeding 5) to form thermodynamically stable salts in the solid state.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “Cₘ₋ₙ” or “Cₘ₋ₙ group” used alone or as a prefix, refers to any group having m to n carbon atoms.

The term “alkyl” represents a linear, branched or cyclic hydrocarbon moiety. The terms “alkenyl” and “alkynyl” represent a linear, branched or cyclic hydrocarbon moiety which has one or more double bonds or triple bonds in the chain. Examples of alkyl, alkenyl, and alkynyl groups include but are not limited to methyl, ethyl, propyl, isopropyl, butyl, isobutyl, sec-butyl, tert-butyl, pentyl, isopentyl, neopentyl, tert-pentyl, hexyl, isohexyl, neohexyl, allyl, vinyl, acetylenyl, ethylenyl, propenyl, isopropenyl, butenyl, isobutenyl, butadienyl, pentenyl, pentadienyl, hexenyl, hexadienyl, hexatrienyl, heptenyl, heptadienyl, heptatrienyl, octenyl, octadienyl, octatrienyl, octatetraenyl, propynyl, butynyl, pentynyl, hexynyl, cyclopropyl, cyclobutyl, cyclopentyl, cyclohexenyl, cyclohexdienyl and cyclohexyl.

Where indicated the “alkyl,” “alkenyl,” and “alkynyl” can be optionally substituted such as in the case of haloalkyls in which one or more hydrogen atom is replaced by a halogen, e.g. an alkylhalide. Examples of haloalkyls include but are not limited to trifluoromethyl, difluoromethyl, fluoromethyl, trichloromethyl, dichloromethyl, chloromethyl, trifluoroethyl, difluoroethyl, fluoroethyl, trichloroethyl, dichloroethyl, chloroethyl, chlorofluoromethyl, chlorodifluoromethyl, dichlorofluoroethyl. Aside from halogens, where indicated, the alkyl, alkenyl or alkynyl groups can also be optionally substituted by, for example, oxo, —NR_{d}Rₑ, —CONR_{d}Rₑ, ═N0-Rₑ, —NR_{d}CORₑ, carboxy, —C(═NR_{d})NRₑR_{f}, azido, cyano, C₁₋₆ alkyloxy, C₂₋₆ alkenyloxy, C₂₋₆ alkynyloxy, —N(R_{d})C(═NRₑ)—NR_{f}R_{g}, hydroxyl, nitro, nitroso, —N(Rₕ)CONRᵢRⱼ, —S(O)_{0.2}Rₐ, —C(O)Rₐ, —C(O)ORₐ, —SO₂NRₐR_{b}, —NRₐSO₂R_{b}, —NRₐSO₂NR_{b}Rₑ, —CRₐN═ORₐ, and/or —NRₐCOOR_{b}, wherein Rₐ-Rⱼ are each independently H, C₁₋₄ alkyl, C₂₋₄ alkenyl, or C₂₋₄ alkynyl. The “alkyl,” “alkenyl,” and “alkynyl” can also be optionally substituted by —OCONRₑR_{f}. The “alkyl,” “alkenyl,” and “alkynyl” can also be optionally substituted by —OCONRₑR_{f} The “alkyl,” “alkenyl,” and “alkynyl” can also be optionally substituted by —C(═S)NR_{d}Rₑ.

As used herein, an “alkylsulfonate” comprises an alkyl, alknenyl or alkynyl moiety linked to a sulfonate group: alkyl-S(O)₂O—, alkenyl-S(O)₂O— or alkynyl-S(O)₂O—. Where indicated, the alkyl, alknenyl or alkynyl can be substituted.

The term “aryl” represents a carbocyclic moiety containing at least one benzenoid-type ring (i.e., may be monocyclic or polycyclic), and where indicated may be optionally substituted with one or more substituents. Examples include but are not limited to phenyl, tolyl, dimethylphenyl, aminophenyl, anilinyl, naphthyl, anthryl, phenanthryl or biphenyl. The aryl groups can be optionally substituted by, for example, halogens, NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₆ alkenyl, C₂₆ alkynyl, C₁₋₆ alkyloxy, C₂₋₆ alkenyloxy, C₂₋₆ alkynyloxy, —S(O)₀₋₂Rₐ, optionally substituted 5-12 member heteroaryl, optionally substituted 6-18 member heteroaralkyl, optionally substituted 3-12 member heterocycle, optionally substituted 4-18 member heterocycle-alkyl, —C(O)Rₐ, —C(O)ORₐ, —SO₂NRₐR_{b}, —NRₐSO₂R_{b}, —NRₐSO₂NR_{b}R_{c}, —CRₐN═OR_{b}, and/or —NRₐCOOR_{b}, wherein Rₐ-Rⱼ are each independently H, C₁₋₄ alkyl, C_{2.4} alkenyl, or C_{2.4} alkynyl. The aryl group can also be optionally substituted by —OCONRₑR_{f}. The aryl group can also be optionally substituted by —C(═S)NR_{d}Rₑ

As used herein, an “arylsulfonate” comprises an aryl moiety linked to a sulfonate group: (aryl —S(O)₂O—). Where indicated, the aryl can be substituted.

The term “heterocycle” represents an optionally substituted, non aromatic, saturated or partially saturated wherein said cyclic moiety is interrupted by at least one heteroatom selected from oxygen (O), sulfur (S) or nitrogen (N). Heterocycles may be monocyclic or polycyclic rings. For example, a 3-12 member heterocycle is an optionally substituted, non aromatic, saturated or partially saturated cyclic moiety having 3-12 ring atoms wherein at least one ring atom is a heteroatom selected from oxygen (O), sulfur (S) or nitrogen (N). Examples include but are not limited to azetidinyl, dioxolanyl, morpholinyl, morpholino, oxetanyl, piperazinyl, piperidyl, piperidino, cyclopentapyrazolyl, cyclopentaoxazinyl, cyclopentafuranyl, tetrahydrofuranyl, tetrahydrothiofuranyl, tetrahydrothiofuranyl, tetrahydropyranyl, tetrahydrothiopyranyl, tetrahydrothiopyranyl dioxyde, thiazolinyl, oxazolinyl, pyranyl, thiopyranyl, aziridinyl, azepinyl, dioxazepinyl, diazepinyl, oxyranyl, oxazinyl, pyrrolidinyl, thiopyranyl, thiolane, pyrazolidinyl, dioxanyl, and imidazolidinyl. Where indicated, the heterocyclic groups can be optionally substituted by, for example, halogens, oxo, —NR_{d}Rₑ, CONR_{d}Rₑ, ═NO—Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRₐR_{b}, C₁₋₆ alkyl, C₂₋₆ alkenyl, C₂₋₆ alkynyl, C₇₋₁₂ aralkyl, C₆₋₁₂ aryl, C₁₋₆ alkyloxy, C₂₋₆ alkenyloxy, C₂₋₆ alkynyloxy, —S(O)₀₋₂Rₐ, C₆₋₁₀ aryl, C₇₋₁₀ aryloxy, C₇₋₁₀ arylalkyl, C₆₋₁₀aryl-C₁₋₁₀alkyloxy, —C(O)Rₐ, —C(O)ORₐ, —SO₂NRₐ, —NRₐSO₂R_{b}, —NRₐSO₂NR_{b}R_{c}, —CRₐN═OR_{b}, and/or —NRₐCOOR_{b}, wherein Rₐ-Rⱼ are each independently H, C₁₋₄ alkyl, C₂₋₄ alkenyl or C₂₋₄ alkynyl. The heterocyclic groups can also be optionally substituted by —OCONRₑR_{f}. The heterocyle group can also be optionally substituted by —C(═S)NR_{d}Rₑ.

The term “heterocycle-alkyl” represents an optionally substituted heterocycle group attached to the adjacent atom by an alkyl, alkenyl, or alkynyl group. It is understood that in a 5-18 member heterocycle-alkyl moiety, the term “5-18 member” represents the total number of ring atoms present in the heterocycle moiety and carbon atoms present in the alkyl, alkenyl or alkynyl portion. Where indicated the heterocycle-alkyl groups can be optionally substituted by, for example, halogens, oxo, —NR_{d}Rₑ, —CONR_{d}Rₑ, —C(═S)NR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRₐR_{b}, C₁₋₆ alkyl, C₂₋₆ alkenyl, C₂₋₆ alkynyl, C₁₋₆ alkyloxy, C₂₋₆ alkenyloxy, C₂₋₆ alkynyloxy, —S(O)₀₋₂Rₐ, C₆₋₁₀ aryl, C₆₋₁₀ aryloxy, C₇₋₁₀ arylalkyl, C₆₋₁₀ aryl-C₁₋₁₀ alkyloxy, —C(O)Rₐ, —C(O)ORₐ, ═NO—Rₑ, —SO₂NRₐR_{b}, —NRₐSO₂R_{b}, —NRₐSO₂NR_{b}R_{c}, —CRₐN═OR_{b}, and/or —NRₐCOOR_{b}, wherein Rₐ—Rⱼ are each independently H, C₁₋₄ alkyl, C₂₋₄ alkenyl or C₂₋₄ alkynyl. The heterocycle-alkyl groups can also be optionally substituted by —OCONRₑR_{f}. The heterocycle-alkyl can also be optionally substituted by —C(═S)NR_{d}Rₑ.

The term “heteroaryl” represents an optionally substituted aromatic cyclic moiety wherein said cyclic moiety is interrupted by at least one heteroatom selected from oxygen (O), sulfur (S) or nitrogen (N). Heteroaryls may be monocyclic or polycyclic rings. For example, a 5-12 member heteroaryl is an optionally substituted, aromatic cyclic moiety having 5-12 ring atoms wherein at least one ring atom is a heteroatom selected from oxygen (O), sulfur (S) or nitrogen (N). Examples include but are not limited to—dithiadiazinyl, furanyl, isooxazolyl, isothiazolyl, imidazolyl, oxadiazolyl, dioxazole, oxatriazole, oxazolyl, pyrazinyl, pyridazinyl, pyrimidinyl, pyridyl, pyrazolyl, pyrrolyl, thiatriazolyl, tetrazolyl, thiadiazolyl, triazolyl, thiazolyl, thienyl, tetrazinyl, thiadiazinyl, triazinyl, thiazinyl, furoisoxazolyl, imidazothiazolyl, thienoisothiazolyl, thienothiazolyl, imidazopyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienopyridyl, benzothienyl, benzoxazinyl, benzothiazinyl, quinazolinyl, naphthyridinyl, quinolinyl, isoquinolinyl, benzopyranyl, pyridopyridazinyl and pyridopyrimidinyl. Where indicated the heteroaryl groups can be optionally substituted by, for example, halogens, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, —S(O)₀₋₂Rₐ, C₆₋₁₀ aryl, C₆₋₁₀ aryloxy, C₇₋₁₀ arylalkyl, C₆₋₁₀aryl-C₁₀alkyloxy, —C(O)Rₐ, —C(O)ORₐ, —SO₂NRₐR_{b}, —NRₐSO₂R_{b}, N—RₐSO₂NR_{b}R_{c}—CRₐN═OR_{b}, and/or —NRₐCOOR_{b}, wherein Rₐ-Rⱼ are each independently H, C₁₋₄ alkyl, C₂₋₄ alkenyl or C₂₋₄ alkynyl. The heteroaryl groups can also be optionally substituted by —OCONRₑR_{f}. The heteroaryl can also be optionally substituted by —C(═S)NR_{d}Rₑ.

The term “heteroaralkyl” represents an optionally substituted heteroaryl group attached to the adjacent atom by an alkyl, alkenyl, or alkynyl group.

The terms “alkoxy,” “alkenyloxy,” and “alkynyloxy” represent an alkyl, alkenyl or alkynyl moiety, respectively, which is covalently bonded to the adjacent atom through an oxygen atom. Examples include but are not limited to methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, isohexyloxy, trifluoromethoxy and neohexyloxy. The terms “aryloxy,” represent an aryl moiety substituted with an oxygen, wherein the point of attachment to the molecule it substitutes is on the oxygen.

The term “haloalkyl” used alone or as a suffix or prefix, refers to a C₁-C₆ alkyl group substituted by 1 to 3 halogen atoms or fluorine up to the perfluoro level. Examples of such groups include trifluoromethyl, tetrafluoroethyl, 1,2-dichloropropyl, 5-bromopentyl, 6-iodohexyl.

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “six-membered” used as prefix refers to a group having a ring that contains six ring atoms.

The term “five-membered” used as prefix refers to a group having a ring that contains five ring atoms.

In addition to the polycyclic heterocycles described above, heterocycle includes polycyclic heterocycles wherein the ring fusion between two or more rings includes more than one bond common to both rings and more than two atoms common to both rings.

Examples of such bridged heterocycles include quinuclidine, diazabicyclo[2.2.1]heptane and 7-oxabicyclo[2.2.1]heptane.

The term “amine” or “amino” refers to —NH₂.

The term “halogen” includes fluorine, chlorine, bromine and iodine.

The term “halogenated,” used as a prefix of a group, means one or more hydrogens on the group are replaced with one or more halogens.

In certain embodiments, one or more compounds of the present description may exist as two or more diastereoisomers (also called “diastereo isomer”) or enantiomers. These two or more diastereo isomers or enantiomers may be isolated using one or more methods described in the present description or other known methods even though the absolute structures and configuration of these diastereo isomers or enantiomers may not be ascertained or determined. In order to identify and/or distinguish these diastereo isomers or enantiomers from each other, designations such as “isomer 1,” “isomer 2,” “diastereo isomer 1,” “diastereo isomer 2,” or “enantiomer 1,” “enantiomer 2” may be used to design the isolated isomers.

One aspect of the present description provides a compound of Formula I (A⁺ X⁻), a diastereoisomer, an enantiomer, or a mixture thereof where the following embodiments are present alone or in combination if applicable:

In one aspect X⁻ is a substituted or unsubstituted arylsulfonate.

In one aspect, X⁻ is a thiocarbamoylarylsulfonate capable of releasing hydrogen sulfide following administration to a patient.

In one aspect, R₂ is substituted or unsubstituted aryl.

In one aspect, R₂ is phenyl unsubstituted or substituted with one to three of C(═S)NRₐR_{b}, —CN, —COOH, C₁-C₆-alkyl, C₁-C₆-alkoxy, halogen, C(═O)NRₐR_{b}, or C₁-C₆ haloalkyl.

In one aspect X is a phenylsulfonate wherein the phenyl is unsubstituted or substituted with —C(═S)NH₂, —COOH, Cl, —CN, or —CH₃.

In one aspect, R₂ is substituted or unsubstituted heteroaryl.

In one aspect, R₂ is a 5- or 6-membered heteroaryl unsubstituted or substituted with one to two of C(═S)NRₐR_{b}, —CN, —COOH, C₁-C₆ alkyl, C₁-C₆-alkoxy, halogen, C(═O)NRₐR_{b}, or C₁-C₆ haloalkyl.

In one aspect, X₋ is 2-thiocarbamoylbenzenesulfonate, 3-thiocarbamoylbenzenesulfonate, 4-thiocarbamoylbenzenesulfonate, 2-cyanobenzenesulfonate, 3-cyanobenzenesulfonate, 2-(carboxylic acid)benzenesulfonate, 3-(carboxylic acid)benzenesulfonate, 4-(carboxylic acid)benzenesulfonate, 4-cholorbenzenesulfonate, 2-carbamoylbenzenesulfonate, 3-carbamoylbenzenesulfonate, 4-carbamoylbenzenesulfonate, p-toluenesulfonate, or p-xylenesulfonate.

In one aspect X⁻ is 2-thiocarbamoylbenzenesulfonate, 3-thiocarbamoylbenzenesulfonate, 4-thiocarbamoylbenzenesulfonate, 2-cyanobenzenesulfonate, 3-cyanobenzenesulfonate, 2-(carboxylic acid)benzenesulfonate, 3-(carboxylic acid)benzenesulfonate, 4-(carboxylic acid)benzenesulfonate, 4-cholorbenzenesulfonate, 3-carbamoylbenzenesulfonate, p-toluenesulfonate, or p-xylenesulfonate.

In one aspect, X⁻ is 2-thiocarbamoylbenzenesulfonate, 3-thiocarbamoylbenzenesulfonate, or 4-thiocarbamoylbenzenesulfonate.

In one aspect, R₂ is substituted or unsubstituted alkyl.

In one aspect, R₂ is a C₁-C₆-alkyl unsubstituted or substituted with one to three of —OH, C(═S)NRₐR_{b}, —CN, —COOH, C₁-C₆-alkoxy, halogen, C(═O)NRₐR_{b}, or C₁-C₆ haloalykl.

In one aspect, X⁻ is a substituted or unsubstituted alkylsulfonate, alkenylsulfonate or alkynylsulfonate

In one aspect, X is a methylsulfonate wherein the methyl is unsubstituted or substituted with —OH.

In one aspect, X is an ethylsulfonate unsubstituted or substituted with —OH.

In one aspect, X⁻ is isethionate, methanesulfonate or ethanesulfonate.

In one aspect, X is:

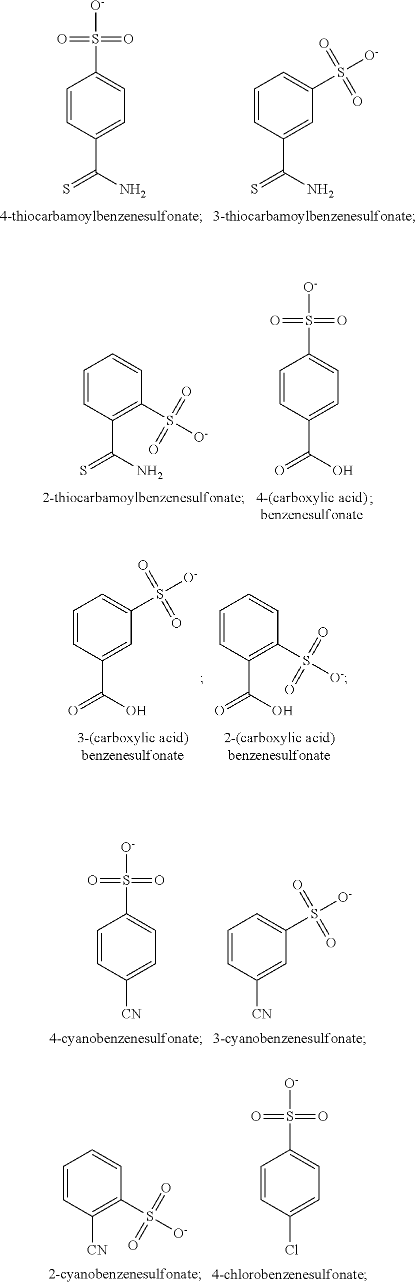

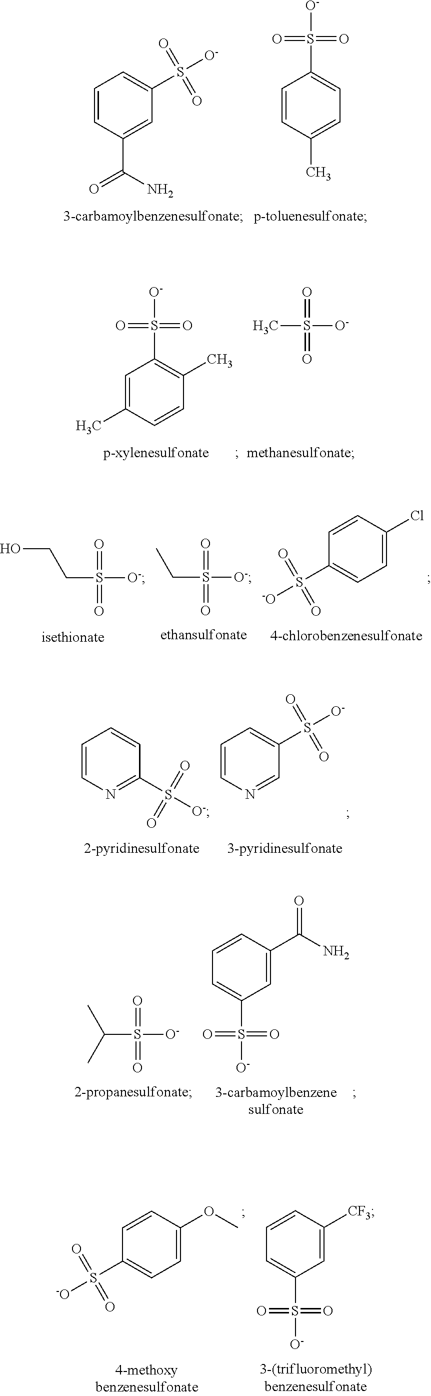

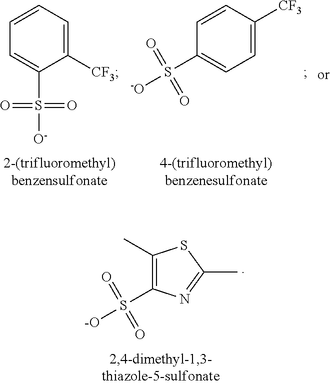

In one aspect, X is:

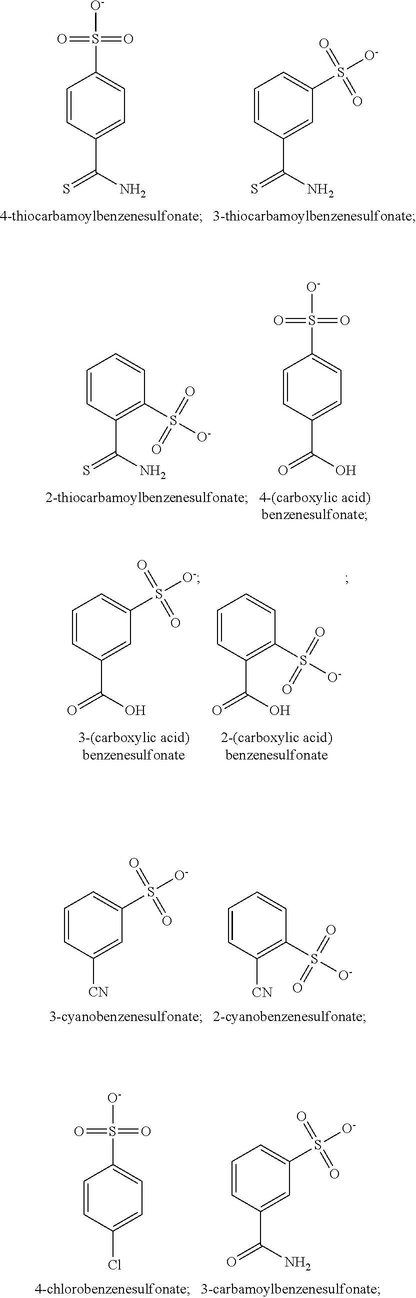

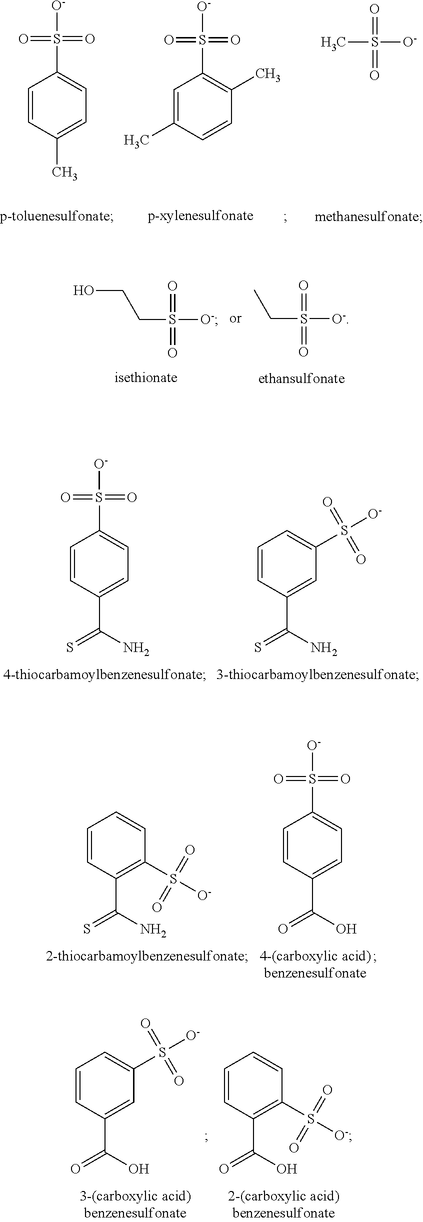

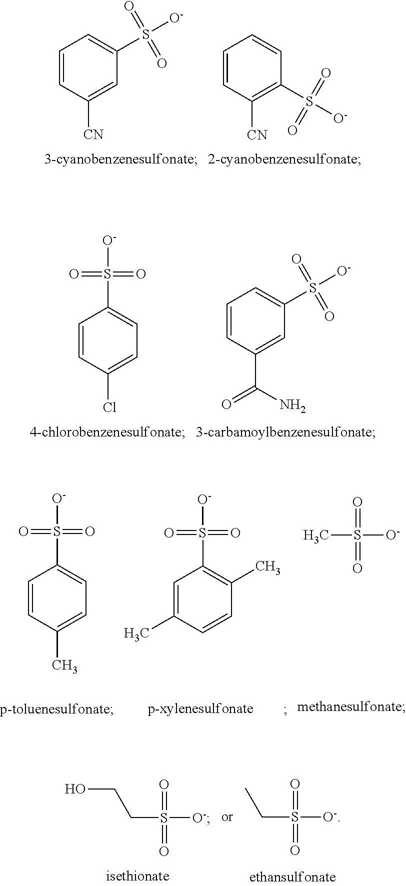

In one aspect, X is:

In one aspect, X⁻ is 2-thiocarbamoylbenzenesulfonate, 3-thiocarbamoylbenzenesulfonate, 4-toluenesulfonate or 4-thiocarbamoylbenzenesulfonate.

In one aspect, X⁻ is 3-thiocarbamoylbenzenesulfonate.

In one aspect, X⁻ is 4-toluenesulfonate.

In one aspect, X⁻ is isethionate, methanesulfonate or ethanesulfonate.

In one aspect, R₁ is hydrogen.

In one aspect, R₁ is methyl.

In one aspect Rₐ and R_{b} are each independently H, C₁₋₄ alkyl, C₂₋₄ alkenyl, or C₂₋₄ alkynyl.

In one aspect Rₐ and R_{b} are each independently H or methyl.

In one aspect Rₐ and R_{b} are H.

One aspect of the present description is a compound of Formula I (A⁺ X⁻), a diastereoisomer, an enantiomer, or a mixture thereof as defined herein wherein the compound is in crystalline form.

One aspect of the present description is a compound of Formula I (A⁺ X⁻), a diastereoisomer, an enantiomer, or a mixture thereof as defined herein wherein the compound is in amorphous form.

In one aspect, there is provided, a pharmaceutical composition comprising at least one compound as defined herein and a pharmaceutically acceptable excipient or carrier.

In one aspect, there is provided a pharmaceutical composition, which is orally, parentally or intrarectally administrable in patients, comprising at least one compound as defined herein and a pharmaceutically acceptable excipient or carrier.

In one aspect, there is provided, a method for reducing visceral pain of a patient, comprising the administration of a visceral pain relieving amount of the pharmaceutical composition as defined herein or of at least one compound as defined herein.

In one aspect, there is provided the use of a compound as defined herein in the preparation of a medicament for reducing visceral pain experienced by a patient.

In one aspect, there is provided the use of a compound or pharmaceutical composition as defined herein for reducing visceral pain experienced by a patient.

In one aspect, the patient is undergoing lower gastrointestinal endoscopy.

In one aspect, the patient is undergoing upper gastrointestinal endoscopy.

In one aspect, the patient is undergoing virtual colonoscopy or barium enema.

In one aspect, the visceral pain is due to gastrointestinal-related diseases.

In one aspect, the gastrointestinal disease (also called gastrointestinal disorder) is all forms of IBS, constipation, a functional gastrointestinal disorder, gastroesophageal reflux disease, functional heartburn, dyspepsia, visceral pain, gastroparesis, chronic intestinal pseudo-obstruction, colonic pseudo-obstruction, Crohn's disease, ulcerative colitis, inflammatory bowel disease, internal and/or external hemorrhoids, radiation proctitis, or other functional disturbances of gastrointestinal motility.

In one aspect, the gastrointestinal-related disease is ulcerative colitis (UC), internal and/or external hemorrhoids, radiation proctitis, all forms of IBS or other functional disturbances of gastrointestinal motility.

In one aspect, the gastrointestinal-related disease is ulcerative colitis (UC), internal and/or external hemorrhoids, radiation proctitis, or all forms of IBS.

In accordance with one aspect of the present description, there is provided a method of synthesizing sulfonic acid derivatives of formula I, including but not limited to, cyanobenzenesulfonic acids, thiocarbamoylbenzenesulfonic acids, thiazolesulfonic acids, pyridylsulfonic acids, trifluorobenzene sulfonic acids, methoxy sulfonic acids, all of which can be separately added to trimebutine in order to form thermodynamically stable salts.

In accordance with one aspect of the present description, there is provided a method of preparing trimebutine or N-desmethyltrimebutine salts of formula I using different alkylsulfonic acid, heteroarylsulfonic acid and arylsulfonic acid derivatives, including but not limited to methanesulfonic acid, ethanesulfonic acid, isethionic acid, p-toluenesulfonic acid, p-xylenesulfonic acid, 4-chlorosulfonic acid, 2-pyridylsulfonic acid, 3-pyridylsulfonic acid, 4-carboxylsulfonic acid, 3-cyanobenzenesulfonic acid, 4-methoxybenzenesulfonic acid, 4-trifluoromethylbenzenesulfonic acid, 3-trifluoromethylbenzenesulfonic acid, 2-trifluoromethylbenzenesulfonic acid, 2,4-dimethyl-1-3-thiazole-5-sulfonic acid, 2-thiocarbamoylbenzenesulfonic acid, 3-thiocarbamoylbenzenesulfonic acid and 4-thiocarbamoylbenzenesulfonic acid, all of which can be separately added to trimebutine in order to form thermodynamically stable salts.

In accordance with one aspect of the present description, there is provided a method of preparing trimebutine salts using different alkylsulfonic acid and arylsulfonic acid derivatives, including but not limited to methanesulfonic acid, ethanesulfonic acid, isethionic acid, p-toluenesulfonic acid, p-xylenesulfonic acid, 2-cyanobenzenesulfonic acid, 3-cyanobenzenesulfonic acid, 2-thiocarbamoylbenzenesulfonic acid, 3-thiocarbamoylbenzenesulfonic acid and 4-thiocarbamoylbenzenesulfonic acid, all of which can be separately added to trimebutine in order to form thermodynamically stable salts.

In accordance with one aspect of the present description, there is provided a method of preparing hydrogen sulfide-releasing trimebutine or N-desmethyltrimebutine salts using different arylsulfonic acid derivatives which, including but not limited to 2-thiocarbamoylbenzenesulfonic acid, 3-thiocarbamoylbenzenesulfonic acid and 4-thiocarbamoylbenzenesulfonic acid, all of which can be added to trimebutine in order to form thermodynamically stable salts.

In accordance with one aspect of the present description, it relates to novel trimebutine salts wherein the counter-ion (anion, X⁻) is selected from one of the following alkylsulfonate, heteroaryl and arylsulfonate derivatives: methanesulfonate, ethanesulfonate, isethionate, p-toluenesulfonate, p-xylenesulfonate, 4-chlorosulfonate, 2-pyridylsulfonate, 3-pyridylsulfonate, 4-carboxylsulfonate, 3-cyanobenzenesulfonate, 4-methoxybenzenesulfonate, 4-trifluoromethylbenzenesulfonate, 3-trifluoromethylbenzenesulfonate, 2-trifluoromethylbenzenesulfonate, 2,4-dimethyl-1-3-thiazole-5-sulfonate, 2-thiocarbamoylbenzenesulfonate, 3-thiocarbamoylbenzenesulfonate acid and 4-thiocarbamoylbenzenesulfonate.

In accordance with one aspect of the present description, there is provided novel trimebutine salts wherein the counterion (anion X⁻) is selected from one of the following alkylsulfonate and arylsulfonate derivatives: methanesulfonate, ethanesulfonate, isethionate, p-toluenesulfonate (known as tosylate), p-xylenesulfonate, 4-chrolobenzenesulfonate, 2-cyanobenzenesulfonate, 3-cyanobenzenesulfonate, 3-carbamoylbenzenesulfonate, 2-thiocarbamoylbenzenesulfonate, 3-thiocarbamoylbenzenesulfonate and 4-thiocarbamoylbenzenesulfonate.

In accordance with one aspect of the present description, there is provided novel trimebutine or N-desmethyltrimebutine salts capable of releasing hydrogen sulfide in-vivo, wherein the counter-ion (anion X⁻) is selected from one of the following arylsulfonate derivatives: 2-thiocarbamoylbenzenesulfonate, 3-thiocarbamoylbenzenesulfonate and 4-thiocarbamoylbenzenesulfonate.

In accordance with one aspect of the present description, there is provided novel trimebutine salts capable of releasing hydrogen sulfide in-vivo, wherein the counterion (anion) is selected from one of the following arylsulfonate derivatives: 2-thiocarbamoylbenzenesulfonate, 3-thiocarbamoylbenzenesulfonate and 4-thiocarbamoylbenzenesulfonate.

In accordance with one aspect of the present description, there is provided the use of trimebutine 3-thiocarbamoylbenzenesulfonate and trimebutine 4-thiocarbamoylbenzenesulfonate and trimebutine 4-toluenesulfonate salts for further profiling to assess properties. Each compound was tested in one or more of the following assays: (1) toxicological evaluation in rodent (mice and rats) and (2) non-rodent species (dogs), (3) in-vitro Caco-2 permeability, (4) in-vitro metabolism over hepatocytes and (5) in-vitro stability in biological fluids.

In accordance with one aspect of the present description, trimebutine, 3-thiocarbamoylbenzenesulfonate could be used in human beings as an analgesic drug for endoscopic applications, such as gastroscopy, colonoscopy and sigmoidoscopy, for medical imaging procedures such as barium enema and virtual colonoscopy, and for the treatment of gastrointestinal disorders, such as hemorrhoids, ulcerative colitis and IBS.

It will be appreciated by those skilled in the art that the compounds can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

It will further be appreciated by those skilled in the art that the compounds in accordance with the present description can exist in different solvate forms, for example hydrates. Solvates of the trimebutine compounds may also form when solvent molecules are incorporated into the crystalline lattice structure of the compound molecule during the crystallization process.

Unless otherwise defined, all technical and scientific terms used herein have the same meaning as commonly understood by one of ordinary skill in the art to which this invention belongs. In addition, the materials, methods, and examples are illustrative only and not intended to be limiting.

For purposes of this present description, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

Additionally, unless otherwise stated, the trimebutine compounds depicted herein are also meant to include compounds that differ only in the presence of one or more isotopically enriched atoms. For example, the trimebutine compounds, wherein one or more hydrogen atoms are replaced deuterium or tritium, or one or more carbon atoms are replaced by a ¹³C- or ¹⁴C-enriched carbon, or one or more sulfur atoms are replaced by a ³⁵S, are within the scope of this present description. Such compounds are useful, for example, as analytical tools, probes in biological assays, or compounds with improved therapeutic profile.

The term visceral pain, as used herein, refers to pain caused by inflammation of serous surfaces, distention of viscera and inflammation or compression of peripheral nerves. Examples of visceral pain include, but are not limited to, abdominal pain, chest pain, pelvic pain, including vulvodynia as well as pain associated with labor or menstruation, and/or pain associated with inflammatory bowel disease, IBS, neurogenic bladder, interstitial cystitis, cholecystitis, pancreatitis and urinary tract infection. In one aspect, the visceral pain is gastrointestinal pain. In one aspect, the visceral pain is associated with inflammatory bowel disease or IBS.

It will be appreciated that the amount of a trimebutine compounds required for use in treatment will vary not only with the particular compound selected but also with the route of administration, the nature of the condition for which treatment is required and the age and condition of the patient and will be ultimately at the discretion of the attendant physician. In general however a suitable dose will be in the range of about 1 to about 30 mg/kg of body weight per day, for example, in the range of 4 to 18 mg/kg/day, or, for example, in the range of 8 to 14 mg/kg/day. Assuming a 70-kg person, such range of doses will represent daily doses of about 70 mg to about 2,100 mg, for example, in the daily dose range of 280 to 1,260 mg/day, or, for example, in daily dose range of 560 to 980 mg/day. As a further example, the daily dose range may also be of 280 to 1,800 mg/day, or, for example, in daily dose range of 560 to 1,500 mg/day.

The desired dose may conveniently be presented in a single dose or as divided dose administered at appropriate intervals, for example as two, three, four or more doses per day.

The trimebutine compound is conveniently administered in unit dosage form; for example containing 25 to 750 mg, 50 to 600 mg, conveniently 75 to 450 mg, most conveniently 125 to 360 mg of active ingredient per unit dosage form. In one embodiment, the trimebutine compound is conveniently administered in unit dosage form of 250 mg.

When trimebutine compounds or pharmaceutically acceptable salts thereof are used in combination with a second therapeutic agent, including but not limited to an anxiolytic drug as a benzodiazepine (e.g. midazolam), an opioid analgesic drug as a fentanyl or meperidine, or an antispasmodic drug as butylscopolamine, the dose of each compound may be either the same as or differ from that when the compound is used alone. Appropriate doses will be readily appreciated by those skilled in the art.

While it is possible that, for use in therapy, the trimebutine compounds may be administered as the raw chemical, it is preferable to present the active ingredient as a pharmaceutical composition. The present description thus further provides a pharmaceutical composition comprising the trimebutine compounds of the present description thereof together with one or more pharmaceutically acceptable carriers therefore and, optionally, other therapeutic and/or prophylactic ingredients. The carrier(s) must be “acceptable” in the sense of being compatible with the other ingredients of the formulation and not deleterious to the recipient thereof.

Pharmaceutical compositions include those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The compositions may, where appropriate, be conveniently presented in discrete dosage units and may be prepared by any of the methods well known in the art of pharmacy. All methods include the step of bringing into association the active with liquid carriers or finely divided solid carriers or both and then, if necessary, shaping the product into the desired composition.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The trimebutine compounds may also be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the trimebutine compounds may be formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Compositions suitable for topical administration in the mouth include lozenges comprising active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical compositions suitable for rectal administration wherein the carrier is a solid are for example presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

Compositions suitable for vaginal administration may be presented as pessaries, tampons, creams, gels, pastes, foams or sprays containing in addition to the active ingredient such carriers as are known in the art to be appropriate.

For intra-nasal administration the compounds or combinations may be used as a liquid spray or dispersible powder or in the form of drops. Drops may be formulated with an aqueous or non-aqueous base also comprising one more dispersing agents, solubilizing agents or suspending agents. Liquid sprays are conveniently delivered from pressurized packs.

For administration by inhalation the compounds or combinations are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the compounds or combinations may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

### DESCRIPTION OF THE FIGURES

FIG. 1 shows X-ray powder diffraction of various lots of Example 20 with either Polymorph A and B.

FIG. 2 shows X-ray powder diffraction of various lots of Example 22 with either Polymorph A, B and C collected on several batches of the compound.

FIG. 3: PK profile of trimebutine following the p.o. administration of trimebutine 3-thiocarbamoylbenzenesulfonate (Example 20).

FIG. 4: PK profile of 3-thiocarbamoylbenzenesulfonate following the p.o. administration of trimebutine 3-thiocarbamoylbenzenesulfonate (Example 20).

FIG. 5: PK profile of trimebutine following the p.o. administration of trimebutine 4-toluenesulfonate (Example 22).

FIG. 6: In vivo efficacy profile of trimebutine 3-thiocarbamoylbenzenesulfonate (Example 20) in the mouse electromyographic colorectal distension induced pain model.

### EXAMPLES

The following examples are merely illustrative of embodiments of the present description, and not limiting to the remainder of this disclosure in any way.

### ABBREVIATIONS

“d” means doublet.
“DCM” means dichloromethane.
“dd” means doublet of doublet.
“DMSO” means dimethylsulfoxide.
“DMSO-d₆” means dimethylsulfoxide-d₆.
“DSC” means differential scanning calorimetry.
“ESI” means electrospray ionization.
“Et” means ethyl.
“EtOAc” means ethyl acetate.
“EtOH” means ethanol.
“Ex” means example.
“g” means gram.
“hr” means hour(s).
“¹H NMR” means proton nuclear magnetic resonance.
“HPLC” means high-performance liquid chromatography.
“IBS” means irritable bowel syndrome.
“IPA” means isopropyl alcohol.
“L” means liter.
“LC” means liquid chromatography.
“LCMS” means liquid chromatography/mass spectroscopy.
“m” means multiplet.
“M” means molar.
“mL” means milliliter.
“μL” means microliter.
“Me” means methyl.
“MeCN” means acetonitrile.
“MeOH” means methanol.
“mg” means milligram.
“MHz” means megahertz.
“min” means minute(s).
“mm” means milimeter.
“μm” means micrometer.
“mmol” means millimole.
“mol” means mole.
“MRM” means multiple reaction monitoring.
“MS” means mass spectrometry.
“MS/MS” and “M2” mean tandem mass spectrometry.
“Pₐₚₚ” means permeability coefficient.
“PK” means pharmacokinetics
“pKₐ” means acid dissociation constant at logarithmic scale.
“ppm” means parts per million.
“Pr” means propyl.
“q” means quartet.
“qt” means quintet.
“rpm” means revolutions per min.
“Rₜ” means retention time (HPLC).
“s” means singlet.
“t” means triplet.
“THF” means tetrahydrofuran
“UV” means ultraviolet.
“VMR” means visceromotor response.
“vol” means volume.
“w/w” means weight over weight.

### Compound Preparation

Examples below illustrate the preparation of the compound of Formula I (A⁺X⁻) and intermediates for making such compounds. It is expected that one skilled in the art of organic synthesis, after reading these examples alone or in combination with the general knowledge in the art, can adapt and apply the methods as desired. The general knowledge in the art includes, for example:
References discussing various organic synthesis reactions, include textbooks of organic chemistry, such as, for example, Advanced Organic Chemistry, March 4th ed, McGraw Hill (1992); and Organic Synthesis, Smith, McGraw Hill, (1994). They also include, for example, R. C. Larock, Comprehensive Organic Transformations, 2nd ed, Wiley-VCH: New York (1999); F. A. Carey; R. J. Sundberg, Advanced Organic Chemistry, 2nd ed., Plenum Press: New York (1984); L. S. Hegedus, Transition Metals in the Synthesis of Complex Organic Molecules, 2nd ed., University Science Books: Mill Valley, Calif. (1994); L. A. Paquette, Ed., The Encyclopedia of Reagents for Organic Synthesis, John Wiley: New York (1994); A. R. Katritzky, O. Meth-Cohn, C W. Rees, Eds., Comprehensive Organic Functional Group Transformations, Pergamon Press: Oxford, UK (1995); G. Wilkinson; F. G A. Stone; E. W. Abel, Eds., Comprehensive Organometallic Chemistry, Pergamon Press: Oxford, UK (1982); B. M. Trost; I. Fleming, Comprehensive Organic Synthesis, Pergamon Press: Oxford, UK (1991); A. R. Katritzky, C W. Rees Eds., Comprehensive Heterocyclic Chemistry, Pergamon Press: Oxford, UK (1984); A. R. Katritzky; C W. Rees, E. F. V. Scriven, Eds., Comprehensive Heterocyclic Chemistry II, Pergamon Press: Oxford, UK (1996); C. Hansen; P. G. Sammes; J. B. Taylor, Eds., Comprehensive Medicinal Chemistry: Pergamon Press: Oxford, UK (1990). In addition, recurring reviews of synthetic methodology and related topics include: Organic Reactions, John Wiley: New York; Organic Syntheses; John Wiley: New York; The Total Synthesis of Natural Products, John Wiley: New York; The Organic Chemistry of Drug Synthesis, John Wiley: New York; Annual Reports in Organic Synthesis, Academic Press: San Diego Calif.; and Methoden der Organischen Chemie (Houben-Weyl), Thieme: Stuttgart, Germany.
References discussing heterocyclic chemistry include, for example, example, Heterocyclic Chemistry, J. A. Joule, K. Mills, G. F. Smith, 3rd ed., Cheapman and Hall, p. 189-225 (1995); and Heterocyclic Chemistry, T. L. Gilchrist, 2^{nd} ed. Longman Scientific and Technical, p. 248-282 (1992).
Databases of synthetic transformations, including Chemical Abstracts, which may be searched using either CAS Online or SciFinder; and Handbuch der Organischen Chemie (Beilstein), which may be searched using SpotFire.

All starting materials in the following compound preparation examples are commercially available or described in the literature. Air and moisture-sensitive liquids and solutions were transferred via syringe or cannula, and introduced into reaction vessels through rubber septa. Reagents and solvents were used without further purification unless otherwise noted.

The terms “concentration under reduced pressure” and “evaporated under reduce pressure” or “concentrated in vacuo” refer to use of a Buchi rotary evaporator at approximately 15 mm of Hg.

¹H NMR spectra were recorded on Varian Gemini 2000 300 MHz.

Melting points were measure using a Differential Scanning Calorimeter (DSC) recorded on DSC Setaram 131.

X-Ray Powder Diffraction (XRPD): The samples were analyzed with Siemens D-5000 diffractometer under following conditions:
every sample was analyzed under ambient conditions using silicon zero background sample holder (low volume specimen holders).
Analysis was performed from 3 to 60 degree 2 theta with Si-Sol-X detector.
Step size 0.01°, step time 1 second
Tube Co K α1,2, divergence slit and receiving of 1 mm each, detector slit 0.1 mm.

### General Procedure 1

### Conversion of a Sulfonyl Chloride Derivative into a Sulfonic Acid Derivative

One molar equivalent of sulfonyl chloride derivative is dissolved in 10 volumes of tetrahydrofuran (THF) and 2.1 molar equivalent of pyridine at room temperature. The solution is cooled down close to 0° C. and one volume of water is added to the solution. The solution is stirred vigorously for about two (2) hr and allowed to warm up to room temperature. Then, solvents are removed under reduced pressure and residual water, if any, was removed by azeotropic distillation using ethanol (approx. 3 volumes). At this stage, the pyridinium sulfonate salt is obtained with 1 molar equivalent of pyridinium hydrochloride. Addition of 4 volumes of ethanol following by filtration gives pure pyridinium sulfonate. Residual pyridine was then removed using Amberlyst®15.

The pyridinium sulfonate salt (5 g) is dissolved in 12 volumes of methanol in which Amberlyst®15 (15 g) is added. The mixture is stirred for 2 hr at room temperature. Amberlyst®15 is removed by filtration and rinsed with 6 volumes of methanol. This last operation can be repeated if residual pyridine is found in filtrate solution. The filtrate contains the sulfonic acid derivative. The sulfonic acid derivative can kept in methanol solution for immediate use, or can be isolated for storage by concentrating the methanol solution under reduced pressure.

### General Procedure 2

### Conversion of a Sulfonate Derivative into a Sulfonic Acid Derivative

Approximately 5 g of sulfonate salt (sodium, potassium or pyridinium) is dissolved in 12 volumes of methanol to which of Amberlyst®15 (15 g) is added. The mixture is stirred for 2 hr at ambient temperature. Amberlyst®15 is removed by filtration and rinsed with 6 volumes of methanol. This last operation is repeated one more time. The filtrate contains the sulfonic acid derivative. The sulfonic acid derivative is kept in methanol solution. The sulfonic acid derivative can kept in methanol solution for immediate use, or can be isolated for storage by concentrating the methanol solution under reduced pressure.

### Example 1

### Synthesis of pyridinium 3-thiocarbamoylbenzenesulfonate

### Step A: Synthesis of pyridinium 3-cyanobenzenesulfonate

3-cyanobenzenesulfonyl chloride (100 g, 0.496 mol) was dissolved in a mixture of THF (1.00 L) and pyridine (82.2 mL, 1.02 mol) at room temperature. The solution was cooled down 0° C. and water (50 mL) was added to the solution. The mixture was stirred vigorously for two (2) hours and left to warm to room temperature. The mixture was then concentrated reduced pressure, and residual water was removed by azeotropic distillation using ethanol (2×1000 mL). Addition ethanol (400 mL), followed by filtration provided the title compound (78 g, 60% yield) as a solid with purity greater than 95%. ¹H-NMR (300 MHz, DMSO-d6): δ 7.58 (t, 1H), 7.82 (dd, 1H), 7.92-7.95 (m, 2H), 8.10 (t, 2H), 8.64 (t, 1H), 8.96 (d, 2H).

### Step B: Synthesis of pyridinium 3-thiocarbamovlbenzenesulfonate

Pyridinium 3-cyanobenzenesulfonate (130 g, 0.496 mol) was dispersed in ethanol (520 mL), and the suspension was slowly transferred to a solution of P₂S₅ (441 g, 1.98 mol) in a mixture of ethanol (880 mL) and hexanes (750 mL). [Note: ethanol (200 mL) was used to rinse the flask and complete the transfer]. The mixture was then stirred at room temperature for 16 hr. Pyridinium 3-thiocarmoylbenzenesulfonate was recovered by filtration as a solid (125 g, 85% yield) with purity greater than 95%. ¹H-NMR (300 MHz, DMSO-d6): δ 7.37 (t, 1H), 7.45 (dd, 2H), 8.09 (t, 2H), 8.19 (t, 1H), 8.62 (t, 1H), 8.95 (d, 2H), 9.59 (s, 1H), 9.89 (s, 1H).

### Example 2

Synthesis of pyridinium 4-thiocarbamoylbenzenesulfonate

The preparation of pyridinium 4-thiocarbamoylbenzenesulfonate was accomplished following a similar procedure to that described for Example 1, replacing pyridinium 3-cyanobenzenesulfonate with pyridinium 4-cyanobenzenesulfonate.

¹H-NMR (300 MHz, DMSO-d₆): δ 7.60 (dd, 2H), 7.82 (dd, 2H), 8.08 (t, 2H), 8.61 (t, 1H), 8.94 (dd, 2H), 9.53 (s, 1H), 9.90 (s, 1H).

### General Procedure 3

### Preparation of a Trimebutine Salt Using a Sulfonic Acid Derivative

Approximately 6.5 g of trimebutine is added to the sulfonic acid derivative solution (1 molar equivalent) in methanol and stirred for one hour at room temperature. The mixture is concentrated under reduced pressure, and acetone (60 mL) is added to the residue. The mixture is then concentrated under reduced pressure, and an additional 60 mL of acetone is added to the residue. The solution is cooled down to 0-5° C. for approximately 2 hr. The title compound crystallizes and the solid is recovered by filtration, washed with cold acetone and put in an oven at 50° C. under nitrogen atmosphere for 16 hr.

### General Procedure 4

### Preparation of Trimebutine Salts Starting with Sulfonic Acid Derivative

Trimebutine (1.93 g, 5.0 mmol) and 5.0 mmol of sulfonic acid are added to 50 mL round-bottom flask. MeOH (20 mL) is added, and the mixture is stirred for 1 hr at room temperature. The resulting solution is divided in 6 equal part parts and transferred to round-bottom flask, and then and each flask is concentrated under reduced pressure. Each residue is then treated using one of the following protocols:
Protocol A: MeOH (5 mL) is added and the mixture is stirred to obtain a solution.
Protocol B: MeOH (5 mL) and water (1 mL) are added, and the mixture is stirred to obtain a solution.
Protocol C: EtOH (5 mL) is added and the mixture is stirred to obtain a solution.
Protocol D: IPA (5 mL) is added and the mixture is stirred to obtain a solution.
Protocol E: Acetone (5 mL) is added and the mixture is stirred to obtain a solution.
Protocol F: Acetone (5 mL) and water (1 mL) is added, and the mixture is stirred to obtain a solution.

Each solution is then transferred into a vial and kept open for the solvent(s) to evaporate at room temperature (18-25° C.) until crystal formation is observed. The solid is then recovered by filtration, washed with solvent and dried under mechanical vacuum.

General procedure 4 was used to prepare Examples 3-9 listed below. Data is reported for the conditions that provided largest quantity of crystals based on visual inspection, although the other conditions attempted may have yielded crystals. Unless otherwise note, the crystallization provided more than 80% yield.

TABLE 1

| | | | Melting | |
|---|---|---|---|---|
| | Starting | | point | |
| | material | Counter-ion name | (DSC | |
| Ex. | (Conditions) | and structure | peaks) | ¹H NMR |
| 3 | Methane sulfonic acid (Protocol E) | 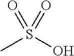 | 181° C. | ¹H NMR (300 MHz, CD₃S(O)CD₃) δ 9.72 (br, 1H); 7.68 (d, J = 6.9 Hz, 2H); 7.48-7.64(m, 3H), 7.23 |
| | | methanesulfonic acid | | (s, 2H); 5.27 (d, J = 13.8 Hz, |
| | | | | 1H); 4.89 (d, J = 13.5 Hz, |
| | | | | 1H); 3.83 (s, 6H); 3.75 (s, |
| | | | | 3H); 2.86 (d, J = 4.8 Hz, |
| | | | | 3H); 2.67 (d, J = 4.8 Hz, |
| | | | | 3H); 2.40-2.55 (m, 1H); |
| | | | | 2.30-2.40 (m, 1H); 2.33 (s, |
| | | | | 3H); 0.75 (t, J = 6.9 Hz, 3H) |
| 4 | p-Xylene sulfonic acid (Protocol A) | 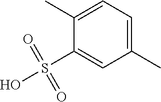 | 143° C. | ¹H NMR (300 MHz, CD₃S(O)CD₃) δ 9.67 (br, 1H); 7.67 (d, J = 6.9 Hz, 2H); 7.48-7.62 (m, 4H), 7.25 (s, 2H); 7.00 (s, 2H); 5.27 (d, J = 13.5 Hz, 1H); 4.89 (d, J = 13.5 Hz, 1H); 3.82 (s, 6H); |
| | | p-xylenesulfonic acid | | 3.75 (s, 3H); 2.86 (d, J = 4.8 |
| | | | | Hz, 3H); 2.67 (d, J = 4.5 Hz, |
| | | | | 3H); 2.40-2.55 (m, 1H); 2.46 |
| | | | | (s, 3H); 2.30-2.40 (m, 1H); |
| | | | | 2.24 (s, 3H); 0.75 (t, J = 7.2 |
| | | | | Hz, 3H). |
| 5 | 4- Chlorobenzene sulfonic acid (Protocol E) | 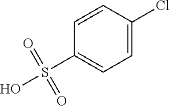 [SMILES: O=S(=O)(O)c1ccc(Cl)cc1] | 131° C. | ¹H NMR (300 MHz, CD₃S(O)CD₃) δ 9.62 (br, 1H); 7.67 (d, J = 6.6 Hz, 2H); 7.50-7.65 (m, 5H); 7.35-7.42 (m, 2H); 7.24 (s, 2H), 5.27 (d, J = 13.2 Hz, 1H); 4.89 (d, J = 13.5 Hz |
| | | 4- | | 1H); 3.83 (s, 6H); 3.75 (s, |
| | | chlorobenzenesulfonic | | 3H); 2.86 (d, J = 4.5 Hz, |
| | | acid | | 3H); 2.67 (d, J = 4.8 Hz, |
| | | | | 3H); 2.40-2.55 (m, 1H); |
| | | | | 2.25-2.40 (m, 1H); 0.76 (t, |
| | | | | J = 7.5 Hz, 3H). |
| 6 | Ethanesulfonic acid (Protocol E) | counter-ion 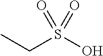 | 184° C. | ¹H NMR (300 MHz, CD₃S(O)CD₃) δ 9.62 (br, 1H); 7.69 (d, J = 7.2 Hz, 2H); 7.50-7.65 (m, 3H), 7.25 (s, 2H); 5.27 (d, J = 13.2 Hz 1H); 4.89 (d, J = 13.5 Hz, |
| | | ethanesulfonic acid | | 1H); 3.83 (s, 6H); 3.75 (s, |
| | | | | 3H); 2.86 (d, J = 4.5 Hz, |
| | | | | 3H); 2.68 (d, J = 4.5 Hz, |
| | | | | 3H); 2.45-2.60 (m, 1H); |
| | | | | 2.30-2.40 (m, 3H); 1.06 (t, |
| | | | | J = 7.2 Hz, 3H); 0.76 (t, J = |
| | | | | 7.2 Hz, 3H). |
| 7 | 2- Pyridinesulfonic acid (Protocol D) | 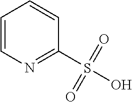 | 131° C. | ¹H NMR (300 MHz, CD₃S(O)CD₃) δ 9.67 (br, 1H); 7.67 (dt, J₁ = 6.6 Hz, J₂ = 0.6 Hz 1H); 7.82 (t, J = 7.2 Hz, 1H); 7.74 (d, J₁ = 7.5 Hz, 1H); 7.68 (d, J = 6.6 Hz, 2H); 7.50-7.64 (m, 3H); |
| | | 2-pyridinesulfonic | | 7.33-7.38 (m, 1H); 7.24 (s, |
| | | acid | | 2H), 5.27 (d, J = 12.3 Hz, |
| | | | | 1H); 4.89 (d, J = 13.5 Hz, |
| | | | | 1H); 3.82 (s, 6H); 3.75 (s, |
| | | | | 3H); 2.87 (br, s, 3H); 2.70 |
| | | | | (br, s, 3H); 2.40-2.55 (m, |
| | | | | 1H); 2.25-2.40 (m, 1H); 0.75 |
| | | | | (t, J = 7.2 Hz, 3H). |
| 8 | 3- Pyridinesulfonic acid (Protocol D) | 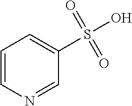 | 119° C., 143° C. | ¹H NMR (300 MHz, CD₃S(O)CD₃) δ 9.64 (br, 1H); 8.77 (s, 1H); 8.53 (d, J = 3.3 Hz, 1H); 7.96 (dt, J₁ = 7.8 Hz, J₂ = 1.8 Hz, 1H); 7.67 (d, J = 6.9 Hz, 2H); 7.50-7.62 (m, 3H); 7.38 (dd, |
| | | 3-pyridinesulfonic | | J₁ = 6.9 Hz, J₂ = 4.5 Hz, 1H); |
| | | acid | | 7.24 (s, 2H), 5.25 (d, J = 13.8 |
| | | | | Hz, 1H); 4.89 (d, J = 13.5 |
| | | | | Hz, 1H); 3.82 (s, 6H); 3.76 |
| | | | | (s, 3H); 2.60-3.00 (br, s, 6H); |
| | | | | 2.40-2.55 (m, 1H); 2.25-2.40 |
| | | | | (m, 1H); 0.75 (t, J = 7.2 Hz, |
| | | | | 3H). |
| 9 | 2- propanesulfonic acid (Protocol C) | 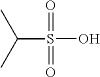 | 136° C. | ¹H NMR (300 MHz, CD₃S(O)CD₃) δ 9.72 (br, 1H); 7.69 (d, J = 7.5 Hz, 2H); 7.50-7.62(m, 3H), 7.24 (s, 2H); 5.27 (d, J = 13.2 Hz, |
| | | 2-propanesulfonic | | 1H); 4.89 (d, J = 13.2 Hz, |
| | | acid | | 1H); 3.83 (s, 6H); 3.75 (s, |
| | | | | 3H); 2.86 (d, J = 4.5 Hz, |
| | | | | 3H); 2.68 (d, J = 4.2 Hz, |
| | | | | 3H); 2.40-2.55 (m, 2H); |
| | | | | 2.25-2.40 (m, 1H); 1.06 (d, |
| | | | | J = 6.9 Hz, 6H); 0.76 (t, J = |
| | | | | 7.2 Hz, 3H). |

Note:
For general procedures 4-6, crystals typically appeared within the first 14 days of evaporation. However, for some examples, samples were left to crystallize for prolonged periods of time (6-9 months), when possible.

### General Procedure 5

### Preparation of Trimebutine Salts Starting from Sodium, Potassium or Pyridinium Sulfonate Derivatives

To a solution of sodium sulfonate (5.0 mmol) in MeOH (20 mL) is added Amberlyst® 15, and the mixture is stirred for 1 hr at room temperature. The resin is removed by vacuum filtration over Celite® and washed with MeOH (5 mL). This process is repeated once. The resulting mixture is passed through a 0.45μ filter, then trimebutine (1.93 g, 5.0 mmol) is added, and the mixture is stirred for 1 hr at room temperature. The resulting solution is divided in 6 equal part parts and transferred to round-bottom flask, and then and each flask is concentrated under reduced pressure. Each residue is then treated using one of the following protocols:
Protocol A: MeOH (5 mL) is added and the mixture is stirred to obtain a solution.
Protocol B: MeOH (5 mL) and water (1 mL) are added, and the mixture is stirred to obtain a solution.
Protocol C: EtOH (5 mL) is added and the mixture is stirred to obtain a solution.
Protocol D: IPA (5 mL) is added and the mixture is stirred to obtain a solution.
Protocol E: Acetone (5 mL) is added and the mixture is stirred to obtain a solution.
Protocol F: Acetone (5 mL) and water (1 mL) is added, and the mixture is stirred to obtain a solution.

Each solution is then transferred into a vial and kept open for the solvent(s) to evaporate at room temperature (18-25° C.) until crystal formation is observed. The solid is then recovered by filtration, washed with solvent and dried under mechanical vacuum.

General procedure 5 was used to prepare Examples 10-12 listed below. Data is reported for the conditions that provided largest quantity of crystals based on visual inspection, although the other conditions attempted may have yielded crystals. Unless otherwise note, the crystallization provided more than 80% yield.

TABLE 2

| | | | Melting | |
|---|---|---|---|---|
| | | Counter-ion | point | |
| | Starting | name and | (DSC | |
| Ex. | material | structure | peaks) | ¹H NMR |
| 10 | 3- Sulfobenzoic acid sodium salt (Protocol A) | 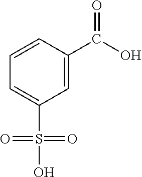 [SMILES: O=C(O)c1cccc(S(=O)(=O)O)c1] | 120, 173° C. (Crystal obtained in MeOH) | ¹H NMR (300 MHz, CD₃S(O)CD₃) δ 9.62 (br, 1H); 7.88 (dd, J₁ = 7.5 Hz J₂ = 1.2 Hz, 1H); 7.86 (dt, J₁ = 7.8, Hz J₂ = 1.2 Hz, 1H); 7.67 (d, J = 7.2 Hz, 2H); 7.52-7.61 (m, 4H), 7.46 (t, J = 7.8 Hz, 1H); 7.24 (s, 2H); 5.27 (d, J = 13.8 Hz 1H); 4.89 (d, J = 13.8 Hz, 1H); 3.83 (s, 6H); 3.76 (s, 3H); 2.86 (d, J = 4.5 Hz, 3H); 2.67 (d, J = 4.5 Hz, 3H); 2.40- 2.55 (m, 1H); 2.30-2.40 (m, 1H); 0.76 (t, J = 7.5 Hz, 3H). |
| | | 3-sulfobenzoic | | |
| | | acid | | |
| 11 | Isethionic acid sodium salt (Protocol E) | 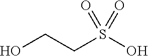 | 164° C. | 1H NMR (300 MHz, CD₃S(O)CD₃) δ 9.62 (br, 1H); 7.67 (d, J = 6.6 Hz, 2H); 7.50-7.65 (m, 3H), 7.24 (s, 2H); 5.27 (d, J = 13.2 Hz 1H); 4.89 (d, J = |
| | | isethionic acid | | 13.5 Hz, 1H); 3.83 (s, 6H); 3.75 (s, |
| | | | | 3H); 3.62 (t, J = 6.6 Hz, 2H); 2.85 (d, |
| | | | | J = 5.1 Hz, 3H); 2.66 (d, J = 4.8 Hz, 3H); |
| | | | | 2.61 (t, J= 6.9 Hz, 2H); 2.40-2.55 (m, |
| | | | | 1H); 2.30-2.40 (m, 1H); 0.76 (t, J = 7.2 |
| | | | | Hz, 3H). |
| 12 | 3-Carbamoyl benzene sulfonic acid potassium salt (Protocol A) | 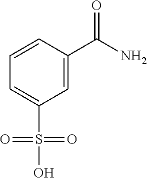 [SMILES: NC(=O)c1cccc(S(=O)(=O)O)c1] | 223° C. | ¹H NMR (300 MHz, CD₃S(O)CD₃) δ 9.75 (br, 1H); 9.19 (br, 1H); 8.16 (t, J = 1.5 Hz, 1H); 8.08 (br, s, 1H); 7.82 (dt, J₁ = 7.5 Hz, J₂ = 1.5 Hz, 1H); 7.76 (dt, J₁ = 7.8 Hz, J₂ = 1.5 Hz, 1H); 7.57- 7.70 (m, 3H), 7.44-7.56 (m, 3H); 7.41 (t, J = 7.5 Hz, 1H); 7.24 (s, 1H); 4.87 (d, J = 12.9, 1H); 4.43 (d, J = 12.9 Hz, 1H); 3.80 (s, 6H); 3.74 (s, 3H); 2.20- 2.80 (m, 8H); 0.72 (t, J = 6.9 Hz, 3H). |
| | | 3- | | |
| | | carbamoylbenzene | | |
| | | sulfonic acid | | |

Note:
For general procedures 4-6, crystals typically appeared within the first 14 days of evaporation. However, for some examples, samples were left to crystallize for prolonged periods of time (6-9 months), when possible.

Example 13 was prepared following General Procedure 5 by substituting trimebutine by N-desmethyl-trimebutine. Data is reported for the conditions that provided largest quantity of crystals based on visual inspection, although the other conditions attempted may have yielded crystals. Unless otherwise note, the crystallization provided more than 80% yield.

TABLE 3

| | | | Melting | |
|---|---|---|---|---|
| | Starting | Counter-ion | point | |
| | material | name and | (DSC | |
| Ex. | (Conditions) | structure | peaks) | ¹H NMR |
| 13 | Pyridinium 3- thiocarbamoyl benzene sulfonate (Protocol A) | 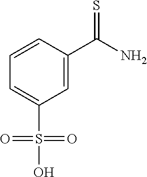 [SMILES: NC(=S)c1cccc(S(=O)(=O)O)c1] | 209° C. | ¹H-NMR (300 MHz, DMSO-d6): δ 9.35 (s, 1H), 9.63 (s, 1H), 9.57 (s, 1H), 8.18 (s, 1H), 7.77 (d, J = 7.8 Hz, 1H), 7.72 (d, J = 7.7 Hz, 1H), 7.68 (m, 2H), 7.53-7.60 (m, 3H), 7.36 (t, J = 7.7 Hz, 1H), 7.25 (s, 2H), 5.27 (d, J = 13.4 Hz, 1H), 4.90 (d, J = 13.4 Hz, 1H), 3.83 (s, 6H), 3.76 (s, 3H), 2.87 (d, J = 4.8 Hz, 3H), 2.68 (d, J = 4.8 Hz, 3H), 2.45-2.51 (m, 1H), 2.33-2.39 (m, 1H), 0.77 (t, J = 7.2 Hz, 3H). |
| | | 3- | | |
| | | thiocarbamoyl | | |
| | | benzene | | |
| | | sulfonic acid | | |

Note:
For general procedures 4-6, crystals typically appeared within the first 14 days of evaporation. However, for some examples, samples were left to crystallize for prolonged periods of time (6-9 months), when possible.

### General Procedure 6

### Preparation of Trimebutine Salts Starting from Sulfonyl Chloride Derivatives

To a solution of sulfonyl chloride derivatives (0.025 mmol) in THF (20 mL) is added pyridine (0.05 mmol). The solution is cooled to 0-5° C. and water (1 mL) is added. The reaction mixture is left to warm to room temperature, stirred for 2 hr, and then concentrated under reduced pressure. To the residue is added EtOH (20 mL), and the mixture is concentrated under reduced pressure. Then EtOH (10) is added to the residue and a suspension is obtained in a majority of trials. The solid is collected by vacuum filtration and dried. Pyridinium sulfonate salt intermediate are obtained with a yield of 50% w/w and more. If no crystal is form, the solution is directly used with Amberlyst-15 without isolation of the pyridinium sulfonate salt.

The solid (pyridinium sulfonate) is then dissolved in MeOH (20 mL) and Amberlyst®15 is added. The mixture is stirred for 1 hr at room temperature, then the resin is removed by filtration on Celite® and washed with MeOH (5 mL). The resulting mixture is passed through a 0.45 m filter, then trimebutine (1 equivalent vs pyridinium sulfonate intermediate) is added, and the mixture is stirred for 1 hr at room temperature. The resulting solution is divided in 6 equal part parts and transferred to round-bottom flask, and then each flask is concentrated under reduced pressure. Each residue is then treated using one of the following protocols:
Protocol A: MeOH (5 mL) is added and the mixture is stirred to obtain a solution.
Protocol B: MeOH (5 mL) and water (1 mL) are added, and the mixture is stirred to obtain a solution.
Protocol C: EtOH (5 mL) is added and the mixture is stirred to obtain a solution.
Protocol D: IPA (5 mL) is added and the mixture is stirred to obtain a solution.
Protocol E: Acetone (5 mL) is added and the mixture is stirred to obtain a solution.
Protocol F: Acetone (5 mL) and water (1 mL) is added, and the mixture is stirred to obtain a solution.

Each solution is then transferred into a vial and kept open for the solvent(s) to evaporate at room temperature (18-25° C.) until crystal formation is observed. The solid is then recovered by filtration, washed with solvent and dried under mechanical vacuum.

General procedure 6 was used to prepare Examples 14-19 listed below. Data is reported for the conditions that provided largest quantity of crystals based on visual inspection, although the other conditions attempted may have yielded crystals. Unless otherwise note, the crystallization provided more than 80% yield.

TABLE 3

| | | | Melting | |
|---|---|---|---|---|
| | Starting | Counter-ion | point | |
| | material | name and | (DSC | |
| Ex. | (Conditions) | structure | peaks) | ¹H NMR |
| 14 | 3-Cyano benzenesulfonic acid (Protocol F) | 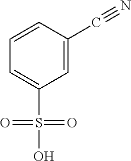 [SMILES: N#Cc1cccc(S(=O)(=O)O)c1] | 129° C. | ¹H NMR (300 MHz, CD₃S(O)CD₃) δ 9.61 (br, 1H); 7.90 (d, J = 6.3 Hz, 2H); 7.79 (d, J = 7.5 Hz, 1H); 7.67 (d, J = 7.2 Hz, 2H); 7.54-7.62 (m, 4H), 7.24 (s, 2H); 5.27 (d, J = 13.5 Hz, 1H); 4.89 (d, J = 13.5 Hz, 1H); 3.83 (s, 6H); 3.76 (s, 3H); 2.85 (d, J = 4.5 Hz, 3 H); 2.67 (d, J = 4.5 Hz, 3H); 2.40-2.55 (m, 1H); 2.30-2.40 (m, 1H); 0.76 (t, J = 6.9 Hz, 3H). |
| | | 3-Cyano | | |
| | | benzenesulfonic | | |
| | | acid | | |
| 15 | 4-Methoxy benzenesulfonyl chloride (Protocol A) | 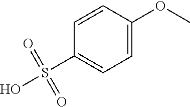 | 165° C. | ¹H NMR (300 MHz, CD₃S(O)CD₃) δ 9.63 (br, 1H); 7.67 (d, J = 6.9 Hz, 2H); 7.48-7.61 (m, 5H); 7.24 (s, 2H), 6.84 (d, J = 6.9 Hz, 2H); 5.27 (d, J = 13.8 Hz 1H); 4.89 (d, J = 13.5 Hz, 1H); 3.82 (s, 6H); 3.76 (s, 3H); 3.75 (s, 3H); 2.85 (d, J = 4.5 Hz, 3H); 2.67 (d, J = 4.5 |
| | | 4-methoxy | | Hz, 3H); 2.40-2.55 (m, 1H); 2.20-2.40 |
| | | benzenesulfonic | | (m, 1H); 0.76 (t, J = 7.2 Hz, 3H). |
| | | acid | | |
| 16 | 3- (Trifluoromethyl) benzenesulfonyl chloride (Protocol E) | 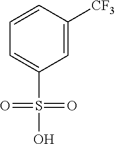 [SMILES: O=S(=O)(O)c1cccc(C(F)(F)F)c1] | 107 and 139° C. | ¹H NMR (300 MHz, CD₃S(O)CD₃) δ 9.62 (br, 1H); 7.85-7.90 (m, 2H); 7.54-7.72 (m, 7H); 7.25 (s, 2H); 5.27 (d, J = 13.8 Hz, 1H); 4.89 (d, J = 13.2 Hz, 1H); 3.83 (s, 6H); 3.76 (s, 3H); 2.86 (d, J = 4.2 Hz, 3H); 2.67 (d, J = 4.8 Hz, 3H); 2.40-2.55 (m, 1H); 2.30-2.40 (m, 1H); 0.76 (t, J = 6.9 Hz, 3H). |
| | | 3- | | |
| | | (trifluoromethyl) | | |
| | | benzenesulfonic | | |
| | | acid | | |
| 17 | 2- (Trifluoromethyl) benzenesulfonyl chloride (Protocol D) | 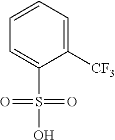 [SMILES: O=S(=O)(O)c1ccccc1C(F)(F)F] | 129° C. | ¹H NMR (300 MHz, CD₃S(O)CD₃) δ 9.61 (br, 1H); 8.07 (d, J = 7.5 Hz, 1H); 7.63-7.70 (m, 3H); 7.40-7.65 (m, 5H); 7.24 (s, 2H), 5.27 (d, J = 13.8 Hz, 1H); 4.88 (d, J = 13.5 Hz, 1H); 3.82 (s, 6H); 3.75 (s, 3H); 2.86 (d, J = 4.8 Hz, 3H); 2.67 (d, J = 4.8 Hz, 3H); 2.40-2.55 (m, 1H); 2.25-2.40 (m, 1H); 0.76 (t, J = 7.2, Hz, 3H). |
| | | 2- | | |
| | | (trifluoromethyl) | | |
| | | benzenesulfonic | | |
| | | acid | | |
| 18 | 4- (Trifluoromethyl) benzenesulfonyl chloride (Protocol D) | 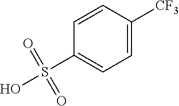 [SMILES: O=S(=O)(O)c1ccc(C(F)(F)F)cc1] | 121° C. | ¹H NMR (300 MHz, CD₃S(O)CD₃) δ 9.61 (br, 1H); 7.67 (d, J = 8.1 Hz, 2H); 7.69 (t, J = 7.5 Hz, 4H); 7.50-7.64 (m, 3H); 7.24 (s, 2H), 5.27 (d, J = 13.5 Hz, 1H); 4.88 (d, J = 13.8 Hz, 1H); 3.83 (s, 6H); 3.75 (s, 3H); 2.86 (d, J = 4.5 Hz, 3H); 2.67 (d, J = 4.8 Hz, 3H); 2.40- |
| | | 4- | | 2.55 (m, 1H); 2.25-2.40 (m, 1H); 0.76 (t, |
| | | (trifluoromethyl) | | J = 7.5 Hz, 3H). |
| | | benzenesulfonic | | |
| | | acid | | |
| 19 | 2,4-dimethyl-l,3- thiazole-5- sulfonyl chloride | 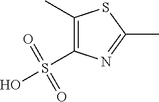 | 155° C. | ¹H NMR (300 MHz, CD₃S(O)CD₃) δ 9.61 (br, 1H); 7.67 (d, J = 7.5 Hz, 2H); 7.50-7.62 (m, 3H); 7.24 (s, 2H), 5.27 (d, J = 13.5 Hz, 1H); 4.88 (d, J = 13.8 Hz, 1H); 3.83 (s, 6H); 3.76 (s, 3H); 2.86 (d, J = 4.8 Hz, 3H); 2.67 (d, J = 4.8 |
| | | 2,4-dimethyl- | | Hz, 3H); 2.40-2.55 (m, 1H); 2.51 (s, |
| | | 1,3-thiazole- | | 3H); 2.25-2.40 (m, 1H); 2.35 (s, 3H); |
| | | 5-sulfonic | | 0.76 (t, J = 6.9 Hz, 3H). |
| | | acid | | |

Note:
For general procedures 4-6, crystals typically appeared within the first 14 days of evaporation. However, for some examples, samples were left to crystallize for prolonged periods of time (6-9 months), when possible.

### Example 20

### Synthesis of trimebutine 3-thiocarbamoylbenzenesulfonate salt

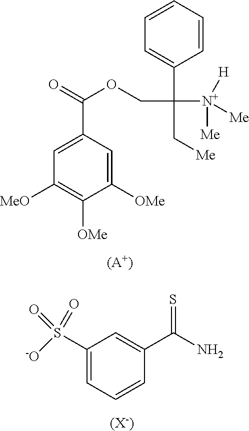

Pyridinium 3-thiocarbamoylbenzenesulfonate (Example 1, 100 g, 0.337 mol) was dissolved methanol (600 mL) to which Amberlyst®15 (200 g) was added. The mixture was stirred for 2 hr at room temperature, then the resin was removed by filtration and rinsed with methanol (approx. 600 mL). This last operation with Amberlyst®15 was repeated until all residual traces of pyridine disappeared from in filtrate (monitored by ¹H-NMR). Once all traces of pyridine had been removed, the filtrate, containing 3-thiocarbamoylbenzensulfonic acid, was used without further characterization.

Trimebutine (130.6 g, 0.337 mol) was added to the methanol solution containing 3-thiocarbamoylbenzensulfonic acid, and the mixture was stirred for one hour at room temperature. The mixture was then concentrated under reduced pressure to a volume of approximately 1 L, then cooled to 0-5° C. and kept at this temperature for about 2 hours. The title product crystallized and was recovered by filtration as a solid. The solid was washed using cold methanol (300 mL) and put in an oven at about 50° C. under nitrogen atmosphere for 16 hr to provide the title compound (173 g, 85% yield) with a purity greater than 95%.

Melting point (by differential scanning calorimetry (DSC) at a temperature ramp of 7° C./min): 183° C.

¹H-NMR (300 MHz, DMSO-d6): δ 9.35 (s, 1H), 9.63 (s, 1H), 9.57 (s, 1H), 8.18 (s, 1H), 7.77 (d, J=7.8 Hz, 1H), 7.72 (d, J=7.7 Hz, 1H), 7.68 (m, 2H), 7.53-7.60 (m, 3H), 7.36 (t, J=7.7 Hz, 1H), 7.25 (s, 2H), 5.27 (d, J=13.4 Hz, 1H), 4.90 (d, J=13.4 Hz, 1H), 3.83 (s, 6H), 3.76 (s, 3H), 2.87 (d, J=4.8 Hz, 3H), 2.68 (d, J=4.8 Hz, 3H), 2.45-2.51 (m, 1H), 2.33-2.39 (m, 1H), 0.77 (t, J=7.2 Hz, 3H).

### Example 21

### Synthesis of 4-thiocarbamoylbenzenesulfonate salt

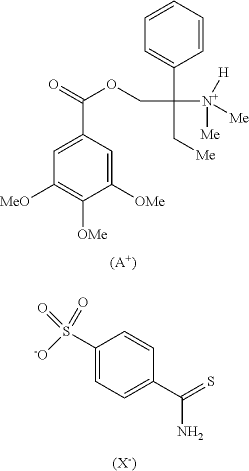

To a solution of pyridinium 4-thiocarbamoylbenzene sulfonate (Example 2, 0.5 g, 1.69 mmol) in MeOH (15 mL) was added Amberlyst®15. The mixture was stirred for 1 hr at room temperature, and then the resin was removed by filtration over Celite® and washed MeOH (5 mL). Amberlyst®15 was then added to the combined filtrates, and the mixture was stirred for 1 hr. The resin was removed by filtration over Celite® and washed MeOH (5 mL). The combined filtrates were then passed through a 0.45μ paper filter (Buchner vaccum filtration) and trimebutine (0.642 g, 1.66 mmol) was then added to the filtrate. The mixture was stirred for 1 hr, and then concentrated under reduced pressure. The residue was then dissolved in a mixture of water (30 mL) and EtOH (10 mL). The resulting mixture was then concentrated under reduced pressure to a volume of 25-30 mL, frozen (using an acetone-dry ice bath at −78° C.) and then lyophilized to yield (0.97 g, 97%) of trimebutine 4-thiocarbamoylbenzene sulfonate as a solid.

Melting point (by differential scanning calorimetry (DSC) at a temperature ramp of 7° C./min): 121° C.

### Example 22

### Synthesis of trimebutine 4-toluenesulfonate salt

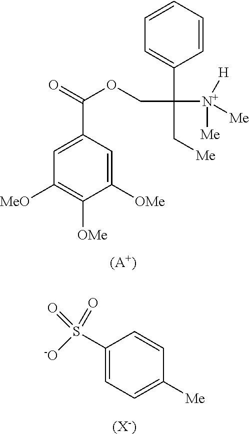

In a 3-L flask, trimebutine (240 g, 0.619 mol) was added followed by EtOH (1.2 L). The mixture was stirred at a temperature ranging from 40-50° C. for 1 hr. Then, a solution of p-toluenesulfonic acid monohydrate (117.8 g, 0.619 mol) in EtOH (480 mL) was added slowly at temperature ranging from 40-60° C. The solution was then heated to 70-75° C. for 1 hr, then cooled to 60-65° C. and seeded with trimebutine p-toluenesulfonate salt. The mixture was then cooled room temperature and stirred for 18 hr. The precipitate was then recovered using a Buchner filter, washed with EtOH (480 mL), and then dried at 40° C. under mechanical vacuum to give the title compound as a solid (319.5 g, 92% yield).

¹H NMR (300 MHz, CD₃S(O)CD₃) δ 9.65 (br, 1H); 7.67 (d, J=6.9 Hz, 2H); 7.54-7.61 (m, 3H); 7.47 (d, J=8.1 Hz, 2H); 7.25 (s, 2H), 7.11 (d, J=7.8 Hz, 2H); 5.27 (d, J=13.5 Hz 1H); 4.89 (d, J=13.5 Hz, 1H); 3.82 (s, 6H); 3.76 (s, 3H); 2.85 (d, J=4.5 Hz, 3H); 2.67 (d, J=4.5 Hz, 3H); 2.40-2.55 (m, 1H); 2.30-2.40 (m, 1H); 2.29 (s, 3H); 0.76 (t, J=6.9 Hz, 3H).

Melting point (by differential scanning calorimetry (DSC) at a temperature ramp of 7° C./min): Three polymorphs have been observed for the title compound: 123, 139, 173° C.

### Polymorphism of trimebutine 3-thiocarbamoylbenzenesulfonate salt (Example 20)

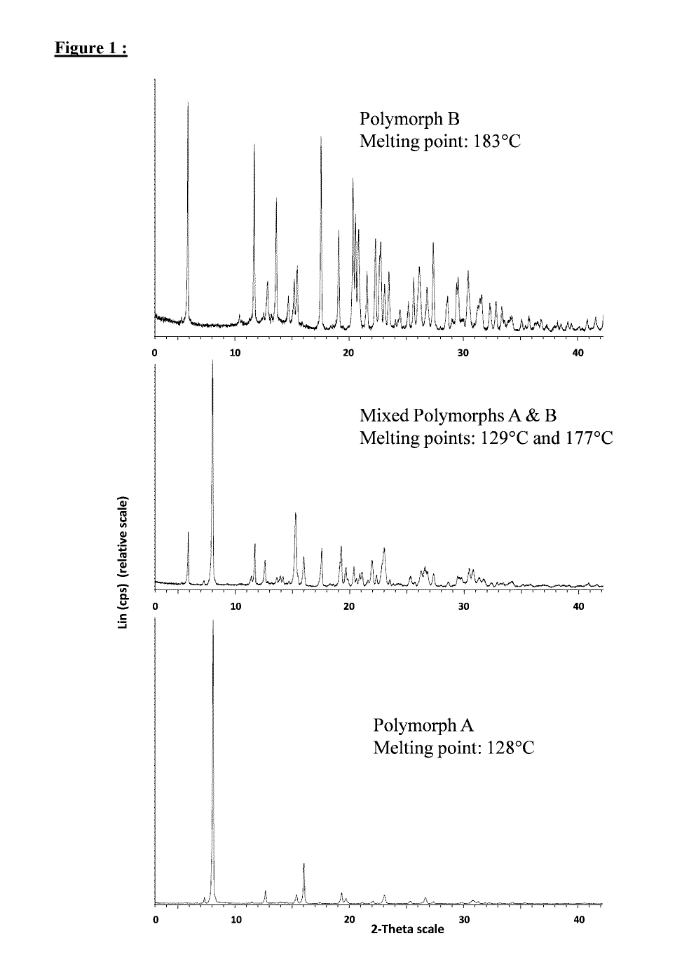

Two different polymorphs of trimebutine 3-thiocarbamoylbenzenesulfonate (Example 21) were identified. Polymorph A was obtained from crystallization in a mixture of acetone and methanol, while Polymorph B was obtained from crystallization in pure methanol. Polymorph B is more stable thermodynamically than Polymorph A based on melting point difference. Polymorph A melts at about 128° C. whereas Polymorph B melts at about 180° C. FIG. 1 shows X-ray powder diffraction of various lots with either Polymorph A and B.

### Polymorphism of trimebutine 4-toluenesulfonate salt

Three different polymorphs of trimebutine p-toluenesulfonate (Example 22) were identified. Polymorph A and B was obtained from crystallization in IPA. Polymorph B was obtained from crystallization in pure ethanol. Polymorph C is also obtained in pure ethanol. Polymorph C is more stable thermodynamically than Polymorph A and B based on melting point difference. Polymorph A melts at about 123° C., polymorph B melts at about 142° C. and Polymorph C melts at about 173° C. FIG. 7 shows X-ray powder diffraction of different lots of trimebutine 4-toluenesulfonate salt showing either Polymorph A, B and C.

### Stability of pyridinium 3-thiocarbamoylbenzenesulfonate and pyridinium 4-thiocarbamoylbenzenesulfonate in various physiological fluids

Pyridinium 3-thiocarbamoyl-benzenesulfonate and pyridinium 4-thiocarbamoyl-benzenesulfonate (100 μM) were separately incubated at 37° C. in simulated gastric fluid (pH 1.2; without pepsin) up to 60 min, in simulated intestinal fluid (pH 6.8, without procreatin) up to 180 min, and in acetate buffer (pH 5) up to 180 min. At time points of 0, 30, 60, 120 and 180 min, an aliquot of 10 μL of samples were removed and added into vials containing 1 M internal standard (labetalol) in a mixture of 25:75 acetonitrile:water. Samples were analyzed by HPLC-MS/MS (ESI-, MRM) to monitor disappearance of its counter-ion over the time. Data suggest that the counterions, 3-thiocarbamoylbenzenesulfonate and 4-thiocarbamoylbenzenesulfonate, were separately stable in all these media over the complete cycle.

### In-vitro metabolism of trimebutine 3-thiocarbamoylbenzenesulfonate over rat, dog and human liver hepatocytes

The evaluation of the metabolic stability of the compound was carried out with pyridinium 3-thiocarbamoylbenzenesulfonate and trimebutine 3-thiocarbamoylbenzenesulfonate using human, dog and rat hepatocytes. Human, dog and rat cryopreserved hepatocytes (Celsis-IVT; n=10 pooled donors) were thawed according to the recommended protocol of the cell provider. Cells were then diluted to 1 million viable cells per mL, plated in a 96-well plate (100 μL per well), and pre-incubated 20 min at 37° C. under 95:5 O₂:CO₂ atmosphere. Following addition of test compounds (10 μM; 1 μL of 1 mM stock solution (95:5 acetonitrile/DMSO) per well), cells were incubated with lid on up to 120 min at 37° C. under 95:5 O₂:CO₂ atmosphere. At time points 0, 15, 60 and 120 min, 100 μL of acetonitrile containing an internal standard (1 μM labetalol) was added to quench incubates, and the plate was centrifuged (5 min; 15K rpm). Supernatant was diluted 1:2 with water and analyzed by HPLC-MS/MS (ESI-). The MS2 scan mode was used instead of the MRM mode, in order to manually extract the Extracted Ion Current (EIC) of two potential metabolites: 3-cyanobenzenesulfonate and sodium 3-sulfobenzoate. During this incubation, 3-thiocarbamoylbenzenesulfonate remained intact during the incubation with hepatocytes from the three species, without observing metabolites. Positive controls, including trimebutine, confirmed that hepatocytes were active and capable of metabolizing trimebutine.

### Permeability of Various Sulfonate Counter-Ions Over Caco-2 Cell Layer

In order to estimate human intestinal permeability and to investigate potential drug efflux, a Caco-2 permeability assay was performed with trimebutine 3-thiocarbamoylbenzenesulfonate, trimebutine 4-toluenesulfonate and different counter-ion candidates to be used in trimebutine salts. Such a procedure helps understanding the suitability of the compound for oral dosing by measuring the rate of transport of the molecule across the Caco-2 cells, which have characteristics that resemble intestinal epithelial cells. Transport in both directions (apical to basolateral (A-B) and basolateral to apical (B-A)) across the cell monolayer was monitored over a 2-hr time period in order to evaluate the efflux ratio, an indicator of whether the compound undergoes significant active efflux or not. The permeability coefficient (Pₐₚₚ) is calculated from the equation:

Pₐₚₚ=(dQ/dt/Cₒ×A)

Where dQ/dt is the rate of permeation of the drug across the cells, Cₒ is the donor compartment concentration at time zero and A is the area of the cell monolayer. Cₒ is obtained from analysis of the dosing solution at the start of the experiment. The analysis method used was LCMS quantification.

TABLE 5
List of compounds tested

| Compounds | Moieties studied |
|---|---|
| Trimebutine 3-thiocarbamoylbenzenesulfonate | Trimebutine, sulfonate salt |
| (trimebutine m/z: 388; counter-ion m/z: 216) | |
| pyridinium 3-thiocarbamoylbenzenesulfonate | Sulfonate counter-ion |
| pyridinium 4-thiocarbamoylbenzenesulfonate | Sulfonate counter-ion |
| pyridinium 3-cyanobenzenesulfonate | Sulfonate counter-ion |
| Sodium 3-sulfobenzoate | Sulfonate counter-ion |
| Trimebutine 4-toluenesulfonate | Trimebutine |
| (trimebutine m/z: 388) | |

TABLE 6
Caco-2 bidirectional permeability results

| | % Recovery | | Pₐₚₚ (×10⁻⁶ cm/s) | | Efflux | Perme- | Significant |
|---|---|---|---|---|---|---|---|
| Test Compound ID | A − B | B − A | A − B | B − A | ratio* | ability | Efflux |
| Pyridinium 4- | 106 | 116 | <0.54 | <0.68 | ND | Low | No |
| thiocarbamoyl- | | | | | | | |
| benzenesulfonate | | | | | | | |
| Pyridinium 3- | 100 | 107 | 0.19 | 0.32 | 1.7 | Low | No |
| thiocarbamoyl- | | | | | | | |
| benzenesulfonate | | | | | | | |
| Trimebutine 4- | 58 | 76 | 23.9 | 21.7 | 0.9 | High | No |
| toluenesulfonate | | | | | | | |
| 3-Thiocarbamoyl- | 63 | 82 | 0.28 | 0.33 | 1.2 | Low | No |
| benzenesulfonate | | | | | | | |
| moiety, as part of | | | | | | | |
| trimebutine 3- | | | | | | | |
| thiocarbamoyl- | | | | | | | |
| benzenesulfonate salt | | | | | | | |
| (counter-ion m/z: | | | | | | | |
| 216) | | | | | | | |
| Trimebutine moiety, | 51 | 62 | 23.2 | 24.3 | 1.0 | High | No |
| as part of trimebutine | | | | | | | |
| 3-thiocarbamoyl- | | | | | | | |
| benzenesulfonate salt | | | | | | | |
| (trimebutine m/z: | | | | | | | |
| 388) | | | | | | | |
| Pyridinium 3-cyano- | 84 | 72 | 0.44 | 0.87 | 2.0 | Low | No |
| benzenesulfonate | | | | | | | |
| Sodium 3- | 93 | 115 | 0.59 | 0.59 | 1.0 | Low | No |
| sulfobenzoate | | | | | | | |

*Efflux ratio = (Pₐₚₚ B − A)/(Pₐₚₚ A − B)

A permeability of (Pₐₚₚ A-B)<1.0×10⁻⁶ cm/s is considered low, whereas a permeability of (Pₐₚₚ A-B)>1.0×10⁻⁶ cm/s is considered high. A significant efflux is generally associated with an efflux ratio above 3.0 and a (Pₐₚₚ B-A)>1.0×10⁻⁶ cm/s in these assay conditions.

The trimebutine moiety of the trimebutine 3-thiocarbamoylbenzenesulfonate and trimebutine p-toluenesulfonate salts showed high permeability, whereas the 3-thiocarbamoylbenzenesulfonate moiety featured a low permeability suggesting that it would be poorly absorbed in-vivo following oral administration. Separately, different counter-ions were evaluated and all arylsulfonate moieties reported in the above Table had poor permeability over Caco-2 cell layer.

### Toxicological Evaluation of Trimebutine 3-Thiocarbamoylbenzenesulfonate Following i.p. Administration in Mice

A preliminary toxicological evaluation of the compound was done in 6-8 week old male Balb/C mice. The animals received a dose of 50 mg/kg of trimebutine 3-thiocarbamoylbenzenesulfonate solubilized in saline by the intraperitoneal (i.p.) route, and after a 2-hr fasting period. Following administration of the test article, animals were observed hr for the first 8 hr post-dosing for clinical signs and twice daily thereafter until termination on Day 7. Body weights were determined on Days 1, 2, 3 and 7. Three mice were sacrificed by exsanguination at 24 hr post dosing or on Day 7, and a gross necropsy was performed in all animals. Particular attention was paid to the abdominal, thoracic and cranial cavities for reporting of any unusual observations. There was no mortality and no clinical signs were noted in any of the mice that received the compound. Body weights remained stable. Moreover, no significant macroscopic finding was found among animals. Consequently, 3-thiocarbamoylbenzenesulfonate, was well-tolerated in mice following i.p. administration at a dose of 50 mg/kg.

### Toxicological and ADME Evaluation of Trimebutine 3-Thiocarbamoylbenzene-Sulfonate Following p.o. Administration in Rats

A preliminary toxicological evaluation of the compound was done in 250-300 g male Sprague-Dawley rats. Animals were randomized into one of three dosing group, i.e. 250, 500 or 1,000 mg/kg administered by oral gavage. Following administration of the test article, animals were observed hourly for the first 8 hr post-dosing for clinical signs and twice daily thereafter until termination on Day 7. Three animals per group were sacrificed by exsanguination under general anesthesia at 24-hr post dosing or on Day 7 and a gross necropsy was performed in all animals. Additionally, all three rats having received the 500 mg/kg dose and scheduled for sacrifice on Day 7 were placed in individual metabolic cages immediately following test article administration, in order to collect feces and urine during 48 hr. Blood samples were collected terminally from each rat for hematology and serum chemistry evaluation.

No mortality or clinical signs were observed in any animals that received the test compound. In general, when administered to male rats at single doses of 250, 500 or 1000 mg/kg, trimebutine 3-thiocarbamoylbenzenesulfonate was well tolerated. Only minor necropsy findings were made in animals sacrificed on Day 7: pale and/or enlarged lungs in a few animals that received either 500 or 1000 mg/kg. The toxicological significance of this finding could not be established. Although some variations compared to control animals were seen in some serum biochemistry parameters (e.g., amylase, creatinine), these variations were minor, transient and remained within the normal range for the species. No significant changes were seen in hematology parameters.

In order to better understand the biodistribution and metabolism of trimebutine 3-thiocarbamoylbenzenesulfonate, the quantification of this compound and its two potential metabolites (3-cyano-benzenesulfonate and 3-sulfobenzoate) was performed in urine and feces collected from the rats dosed with 500 mg/kg of the compound. After proper urine and feces samples preparation, trimebutine 3-thiocarbamoyl-benzenesulfonate and its two metabolites were assayed by HPLC-MS/MS (ESI-), in MRM mode. Calibration curves were prepared for each analyte, in both matrices (diluted blank urine and diluted blank feces).

In average, a quasi-quantitative recovery was obtained for the unchanged 3-thiocarbamoylbenzenesulfonate counter-ion, meaning that the majority of counter-ion was recovered in urine and feces. Nevertheless, 3-cyanobenzenesulfonate was found in feces representing about 4% of the administered dose. Some traces of 3-sulfobenzoate were found (0.3% of administered dose). About 80% of the total amount of counter-ion administered orally was found in feces, versus 20% in urine. This data strongly suggests that 3-thiocarbamoylbenzenesulfonate counter-ion is poorly absorbed in-vivo and a poor oral bioavailability is expected. Most of this molecule resided in the intestinal tract, where the present of metabolites highly suggests that H₂S gaseous mediator was released in the gastrointestinal lumen.

Moreover, an additional toxicology study was carried out in male and female Sprague Dawley rats, in order to establish the maximum tolerated dose (MTD) of trimebutine 3-thiocarbamoylbenzenesulfonate in this animal species. Consequently, a single dose acute and 7-day range-finding oral toxicity study in Sprague-Dawley rats was conducted. During the single dose acute phase of the study, the test article was administered as a single dose by oral gavage to groups of 3 male and 3 female Sprague-Dawley rats, each group receiving a higher or lower dose level, based on the reaction of the previous group during the first day of the observation period. During the range-finding phase, trimebutine 3-thiocarbamoylbenzenesulfonate was administered once daily for 7 consecutive days by oral gavage to groups of 5 male and 5 female Sprague-Dawley rats.

Upon completion of the 7-day treatment period, all animals were euthanized and subjected to a gross necropsy examination (Day 8 study). Clinical signs (ill health, behavioral changes, etc.) were recorded on all surviving animals. Clinical pathology evaluations (hematology, clinical chemistry and coagulation parameters) were performed on all surviving animals (single dose acute & range-finding phases) prior to their scheduled necropsy. Blood samples were collected terminally from the abdominal aorta (while anesthetized with isoflurane).

The results obtained showed that a single dose of trimebutine 3-thiocarbamoylbenzenesulfonate was well-tolerated up to 2,000 mg/kg in both male and female rats. There was no death and no significant clinical signs of toxicity. However, during the 7-day repeat dose regimen, two animals from the 2,000 mg/kg dose group died, respectively, after 3 and 5 days of repeated dosing. Cause of death was unknown, and there was no macroscopic abnormality observed at necropsy. Nevertheless, both male and female rats well tolerated 1,000 mg/kg over 7 days without significant clinical signs of toxicity.

### Pharmacokinetic and ADME Evaluation of Trimebutine 3-Thiocarbamoyl-Benzenesulfonate Following i.v and p.o. Administration in Dogs

In order to evaluate the absolute bioavailability of trimebutine 3-thiocarbamoylbenzenesulfonate, an in-vivo pharmacological study was carried out in six Beagle dogs which were randomized to either 2 mg/kg of the compound administered by the intravenously (i.v.) route, or 10 mg/kg by the oral gavage route (p.o.). Trimebutine 3-thiocarbamoyl-benzenesulfonate was administered as a cross over design once, each dosing separated by at least a 7-day washout period. During this study, assessments included mortality checks, clinical observations, and body weights. Feces and urine samples were collected up to 48 hr post-administration. Blood samples were also collected for pharmacokinetic evaluations on Days 1 and 8 at 10 time points. Pharmacokinetic data are presented in the two following Figures.

From a toxicological assessment point of view, there was no death reported, no change in bodyweight and no indication of any toxicity. Doses administered were low but considered to be pharmacologically active, based on published data on the effects of the trimebutine moiety of the compound.

After proper preparation of the urine and feces samples, 3-thiocarbamoyl-benzenesulfonate counter-ion and its two potential metabolites (3-cyanobenzenesulfonate and 3-sulfobenzoate) were assessed using a LC/MS/MS method, with analysis by MRM, ESI-. Calibration curves were prepared for each analyte in blank feces homogenate, following the same procedure. The mean total recovery of the unchanged counter-ion was about 61% after p.o. administration of trimebutine 3-thiocarbamoylbenzenesulfonate, and about 72% after i.v. administration.

Following oral administration of trimebutine 3-thiocarbamoylbenzenesulfonate, an additional 14.5% recovery was the metabolite, 3-cyanobenzenesulfonate, and another 1.7% recovery was associated to 3-sulfobenzoate. The latter was not detected in urine, strongly suggesting that it is formed exclusively in the gastrointestinal lumen.

Following i.v. administration of trimebutine 3-thiocarbamoylbenzenesulfonate, an additional 4.7% recovery was the metabolite, 3-cyanobenzenesulfonate, and another 0.6% recovery was associated to 3-sulfobenzoate. This observation strongly suggests that the conversion of 3-thiocarbamoylbenzenesulfonate counter-ion predominantly takes place in the gastrointestinal lumen, mainly follows the cyano derivative pathway, and produces in situ relase of H₂S in the gastrointestinal tract.

### Toxicological Evaluation of Trimebutine 3-Thiocarbamoylbenzenesulfonate Following p.o. Administration in Dogs

A second toxicology study was carried out in beagles dogs, in order to determine the MTD of trimebutine 3-thiocarbamoylbenzenesulfonate in this animal species, as well as a 7 day dose range finding. The objectives of this study were (a) to determine the MTD, following five (5) escalating doses to two Beagle Dogs administered as oral gavage (100 to 2,000 mg/kg, until the maximum tolerated dose is considered to have been reached, and (b) to determine the toxicity of trimebutine 3-thiocarbamoylbenzenesulfonate, during a 14-day observation period, following a single oral gavage administration at the MTD in two Beagle Dogs.

For MTD determination, the compound was administered once on each occasion by oral gavage, in an incremental fashion to animals, until the maximum tolerated dose is considered to have been reached. This dose was established at 2,000 mg/kg, i.e., the highest dose administered in animals. The following clinical signs were noted in the female dog: Decreased activity level, vomiting, bowel movement, yellowish fluid fecal consistency, vocalization about 15-20 min post-dose, decreased respiration, weak behavior, eyes partially closed, and mild transient tremors. The animal was back to normal activity levels approximately 1 hr post dosing. There was almost no change in blood pressure approximately 30 min post dose. For one male dog, clinical observations were limited to vomiting a few min following dosing administration. I suspect that the animal did not absorb the full amount of formulation due to the vomiting. The blood pressure dropped slightly at 30 min post dose compared to pre-dose blood pressure values. Overall, Beagle dogs well tolerated doses orally administered up to 2,000 mg/kg of trimebutine 3-thiocarbamoylbenzenesulfonate.

### Pharmacokinetic Evaluation of Trimebutine 4-Toluenesulfonate (Example 22) Following p.o. Administration in Rats

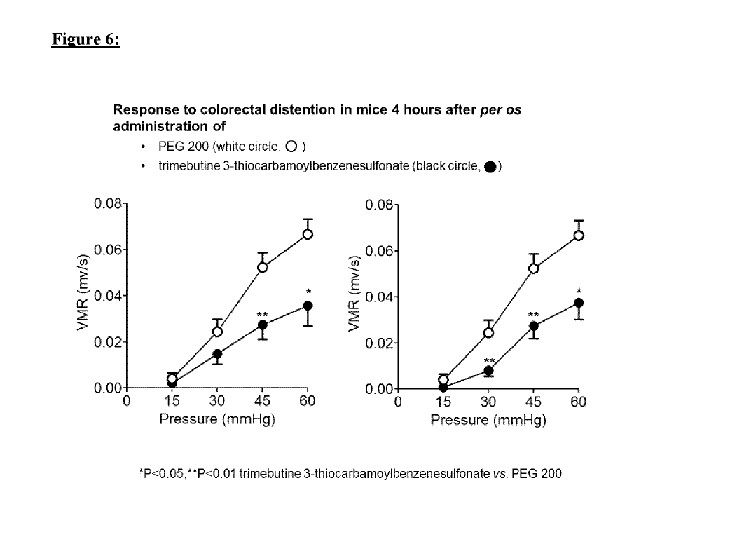

In order to evaluate the absolute bioavailability of trimebutine 4-toluenesulfonate (Example 22), an in-vivo pharmacological study was carried out in 6 Sprague-Dawley rats, which were administered a single dose of 230 and 460 mg/kg of the compound by the oral gavage route (p.o.). During this study, assessments included mortality checks and clinical observations. Blood samples were also collected for pharmacokinetic evaluations at 10 time points. Pharmacokinetic data are presented in FIG. 5.

From a toxicological assessment point of view, there was no death reported, no change in bodyweight and no indication of any toxicity. Doses administered were low but considered to be pharmacologically active, based on published data on the effects of the trimebutine moiety of the compound.

After proper preparation of the plasma samples, trimebutine, N-desmethyltrimebutine and 3,4,5-trimethoxybenzoic acid were assessed using a LC/MS/MS method, with analysis by MRM, ESI-. Calibration curves were prepared for each analyte using standard procedures.

### Development of an Adequate Oral Solid Dosage Form of Trimebutine 3-Thiocarbamoylbenzenesulfonate

### Example of Direct Compression (DC)

A lot was prepared using a dry blending direct compression technique. Ingredients a) to e) in the following Table were sieved using a 30 mesh screen and mixed for 5 min at 25 rpm in a 250 ml V-blender shell (PK Blendmaster). The lubricant was added (item f) to the blender and mixed for 2 min at 25 rpm.

TABLE
Lot produced by Direct Compression (DC) Formulation

| Item | Ingredient name | % w/w | mg/unit | g/batch |
|---|---|---|---|---|
| A | trimebutine | 20.84 | 125.0 | 4.17 |
| | 3-thiocarbamoylbenzenesulfonate | | | |
| B | Lactose monohydrate | 37.58 | 225.5 | 7.52 |
| C | Microcrystalline cellulose type 102 | 36.08 | 216.5 | 7.22 |
| D | Sodium starch glycolate | 3.00 | 18.0 | 0.60 |
| E | Colloidal silicon dioxide | 1.50 | 9.0 | 0.30 |
| F | Magnesium stearate | 1.00 | 6.0 | 0.20 |

| | | | |
|---|---|---|---|
| Core Total: | 100.00 | 600.0 | 20.0 |

### Example of Dry Granulation (DG)

A lot was prepared using a dry granulation approach based on slugging as per next Table. The internal phase ingredients (except magnesium stearate) were first sieved on a 30 mesh screen and mixed using a V-blender for 5 min at 25 rpm. The intra-granular magnesium stearate was added and mixed for 2 min. This mixture was used to create slugs at various forces using a hydraulic press (Carver Model C) with 12 mm round standard concave tooling. The slugs were then crushed using mortar/pestle and sieved through a 20 mesh screen (850 μm opening). The external phase ingredient weight was adjusted according to dry granulation yield. Afterward, internal and external phase were mixed for 2 min using a V-blender at 25 rpm.

TABLE
Lot produced by Dry Granulated (DG) Formulation

| Item | Ingredient name | % w/w | mg/unit | g/batch |
|---|---|---|---|---|
| A | trimebutine | 20.84 | 125.0 | 4.17 |
| | 3-thiocarbamoylbenzenesulfonate | | | |
| B | Lactose monohydrate | 37.58 | 225.5 | 7.52 |
| C | Microcrystalline cellulose type 102 | 36.58 | 219.5 | 7.32 |
| D | Sodium starch glycolate | 3.00 | 18.0 | 0.60 |
| E | Colloidal silicon dioxide | 0.50 | 3.0 | 0.10 |
| F | Magnesium stearate | 0.50 | 3.0 | 0.10 |
| G | Microcrystalline cellulose type 102 | 0.50 | 3.0 | 0.10 |
| H | Magnesium stearate | 0.50 | 3.0 | 0.10 |

| | | | |
|---|---|---|---|
| Core Total: | 100.00 | 600.0 | 20.0 |

Wet Granulation (WG)

A lot was prepared using the wet granulation approach as described in the next Table. The internal phase ingredients were first sieved on a 30 mesh screen and mixed using mortar/pestle for 2 min. The blend was granulated using 4.0 g of purified water (20% on dry basis) as granulation liquid. The water was slowly added using a pipette during approximately 1.5 min. The total granulation time was 2.5 min. The wet mass was dried in a tray oven (Thelco model 18) for 2 hr at a temperature of 50° C. After an overnight at room temperature, the dry material was sieved through a 20 mesh screen. The external phase ingredient weight was adjusted according to dried granulation yield. Afterward, internal and external phase were mixed for 2 min using a V-blender at 25 rpm.

TABLE
Lot produced by Wet Granulation (WG) Formulation

| Item | Ingredient name | % (w/w) | mg/unit | g/batch |
|---|---|---|---|---|
| a | trimebutine | 20.84 | 125.0 | 4.17 |
| | 3-thiocarbamoylbenzenesulfonate | | | |
| b | Lactose monohydrate | 36.58 | 219.5 | 7.32 |
| c | Microcrystalline cellulose type 102 | 36.58 | 219.5 | 7.32 |
| d | Sodium starch glycolate | 3.00 | 18.0 | 0.60 |
| e | Povidone type K29/32 | 1.50 | 9.0 | 0.30 |
| f | Purified Water | 20.4 | Not | 4.01 |
| | | | determined | |
| g | Microcrystalline cellulose type 102 | 0.50 | 3.0 | 0.10 |
| h | Magnesium stearate | 1.00 | 6.0 | 0.20 |

| | | | |
|---|---|---|---|
| Core Total: | 100.00 | 600.0 | 20.0 |

### Stability Assessment of Trimebutine 3-Thiocarbamoylbenzenesulfonate (Example 20, Polymorph B)

The long-term stability of Example 20 was assessed using an accelerated stability protocol. For this purpose, samples of Example 20—Polymorph B was placed in borosilicate vials polyethylene plastic bags, sealed in an aluminum bag, and placed in a fiber drum with desiccant. The samples where then subjected to 40±2° C. with 75±5% relative humidity (RH), and stability was monitored at time 0, 1, 2, 3 and 6 months using a standardized HPLC method. After six months, no degradation had been observed (HPLC purity >99.7%).

### Evaluation of Antinociceptive Properties of Trimebutine 3-Thiocarbamoylbenzenesulfonate (Example 20) in the Mouse CRD Induced Pain Model

The purpose was to evaluate the antinociceptive effects of Example 20 in an electromyographic colorectal distension (CRD) induced pain model. This model is recognized by experts in the field to be less subjective than the rat Abdominal Withdrawal Response (AWR) colorectal distension model, and has been used to assess the analgesic and sedative properties of several new compounds, including IBS drugs Zhuo, M; Gebhart, G. F. Facilitation and attenuation of a visceral nociceptive reflex from the rostroventral medulla in the rat. Gastroenterology, 2002, 122 1007-1019; Larsson, M. H.; Rapp, L.; Lindström, E. Effect of DSS-induced colitis on visceral sensitivity to colorectal distension in mice. Neurogastroenterology & Motility, 2006, 18, 144-152; Arvidsson, S.; Larsson, M.; Larsson, H.; Lindström, E.; Martinez, V. J. Pain, 2006, 7, 108-118; Jones, R. C. W.; Gebhart, G. F. Models of Visceral Pain: Colorectal Distension (CRD). Currrent Protocols in Pharmacology, 2004, 5.36, DOI: 10.1002/0471141755.ph0536s25.]

A brief description of the experimental conditions used is provided below:

[For a complete description of the protocol, see: Cenac, N. et al J Clin Invest. 2007; 117(3):636-647].

### Experimental Design

Three groups of 10 male mice (C57Bl6) were used: 2 groups received treatments with Example 21 and one group received their vehicle (0.9% saline).

The groups were divided as follows:
i. 2 groups of 10 mice received an oral administration of Example 20 at two doses, 30 and 60 mg/kg.
ii. 1 group of 10 mice received an oral administration of the vehicle (PEG 200) use to administer Example 20.

### Experimental Protocol

All groups were implanted with electrodes in the abdominal external oblique musculature under anaesthesia. The surgery for electrode implantation was performed 5 days before the day of the experiment. The day of the experiment, colorectal distension (CRD) was performed in all animals, by inserting a balloon (10-mm long) into the colon, at 5 mm from the anus. The balloon was inflated with warm water, in a stepwise manner, from 0 to 60 mm Hg, with 15 mm Hg increments. Ten-seconds distension periods were performed at pressures of 15, 30, 45 and 60 mm Hg, with 5-min intervals, as previously described, [Al-Chaer et al., A New Model of Chronic Visceral Hypersensitivity in Adult Rats Induced by Colon Irritation During Postnatal Development. Gastroenterology 2000; 119: 1276-1285;] and electromyography recordings were performed during those periods. At the end of all those measures of basal nociceptive response to CRD, groups of mice received their respective treatments. At various times after these treatments: 2, 4 and 6-hours, the same series of stepwise CRD were performed and electromyographic responses (VMR in millivolts/sec) were recorded.

Results of this study are described below:
i. Prior to IP administration of either Example 20 or the saline control, a significant visceromotor response (VMR) to colorectal distension was observed at all 4 pressure levels used. Moreover, an apparent dose-effect response was seen in mice. These preliminary measurements were done to validate the pain inducing effects of colorectal distension, according to the established model parameters.
ii. When Example 20 was compared to the control group, there was an overall reduction of the VMR response after 2 and 4 hours. There was a trend toward efficacy at 6 hours, however the effect was not statistically different at high levels of significance from that of the control group, suggesting a transient antinociceptive effect of Example 20 in this model

These results show that Example 20 exerts significant antinociceptive effects on colorectal distension induced pain the VMR mouse model.

What is claimed:

1. A compound of Formula I (A⁺X⁻), a diastereoisomer, an enantiomer, or a mixture thereof: 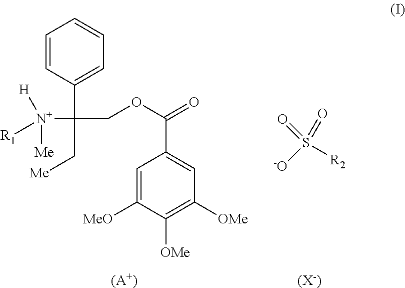
wherein:
R₁ is hydrogen or methyl; and
R₂ is phenyl unsubstituted or substituted with one to three of C(═S)NRₐR_{b}, —CN,
—COOH, C₁-C₆ alkyl, C₁-C₆ alkoxy, halogen, C(═O)NRₐR_{b}, or C₁-C₆haloalkyl, wherein Rₐ and R_{b} are each independently H, C₁₋₄ alkyl, C₂₋₄ alkenyl, or C₂₋₄ alkynyl; or
wherein R₂ is a C₁-C₆ alkyl unsubstituted or substituted with one to three of —OH, C(═S)NRₐR_{b}, —CN, —COOH, C₁-C₆-alkoxy, halogen, C(═O)NRₐR_{b}, or C₁-C₆ haloalkyl.

2. The compound of claim 1, wherein R₂ is phenyl unsubstituted or substituted with one to three of C(═S)NRₐR_{b}, —CN, —COOH, C₁-C₆ alkyl, C₁-C₆ alkoxy, halogen, C(═O)NRₐR_{b}, or C₁-C₆ haloalkyl, wherein Rₐ and R_{b} are each independently H, C₁₋₄ alkyl, C₂₋₄ alkenyl, or C₂₋₄ alkynyl.

3. The compound of claim 1, wherein X is a phenylsulfonate wherein the phenyl is unsubstituted or substituted with —C(═S)NH₂, —COOH, Cl, —CN, or —CH₃.

4. The compound of claim 1, wherein X is ethylsulfonate unsubstituted or substituted with —OH.

5. The compound of claim 1, wherein X is:

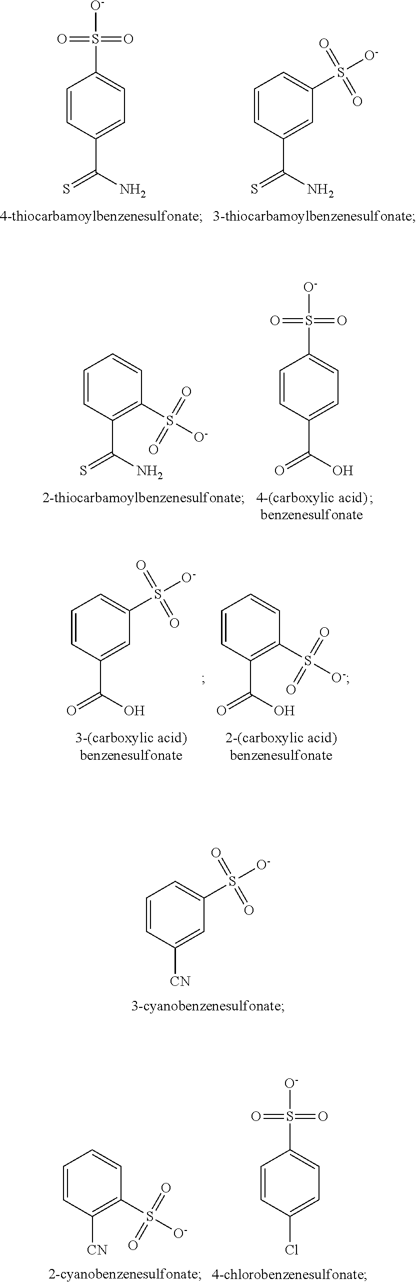

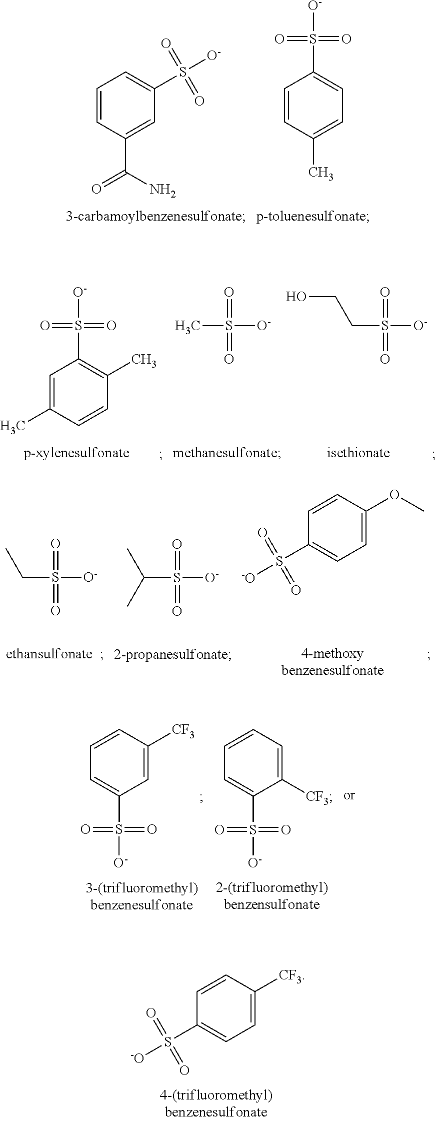

6. The compound of claim 1, wherein X⁻ is 2-thiocarbamoylbenzenesulfonate, 3-thiocarbamoylbenzenesulfonate, 4-toluenesulfonate or 4-thiocarbamoylbenzenesulfonate.

7. The compound of claim 1, wherein X⁻ is 3-thiocarbamoylbenzenesulfonate.

8. The compound of claim 1, wherein X⁻ is 4-toluenesulfonate.

9. The compound of claim 1, wherein X⁻ is isethionate, methanesulfonate or ethanesulfonate.

10. The compound according to claim 1, wherein R₁ is hydrogen.

11. The compound according to claim 1, wherein R₁ is methyl.

12. The compound of claim 1, which is a crystalline polymorph of trimebutine 3-thiocarbamoylbenzenesulfonate salt.

13. The compound of claim 12, wherein the polymorph has a melting point of about 180° C.

14. The compound of claim 12, wherein the polymorph has an X-ray powder diffraction pattern substantially as shown in FIG. 1 as polymorph B.

15. The compound of claim 1, which is a crystalline polymorph of trimebutine p-toluenesulfonate.

16. The compound of claim 15, wherein the polymorph has a melting point of about 173° C.

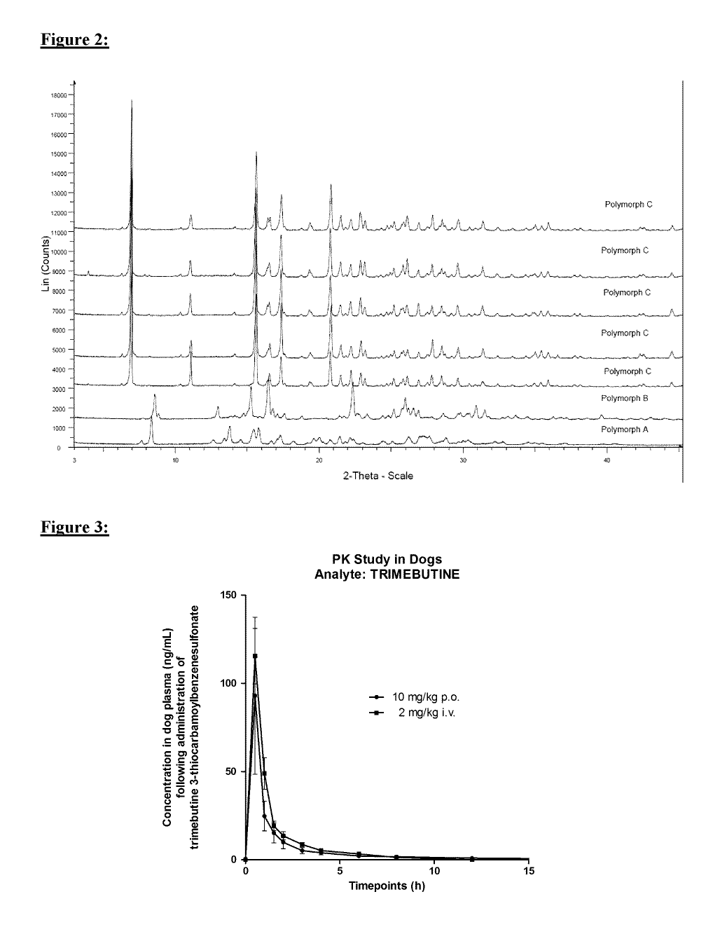

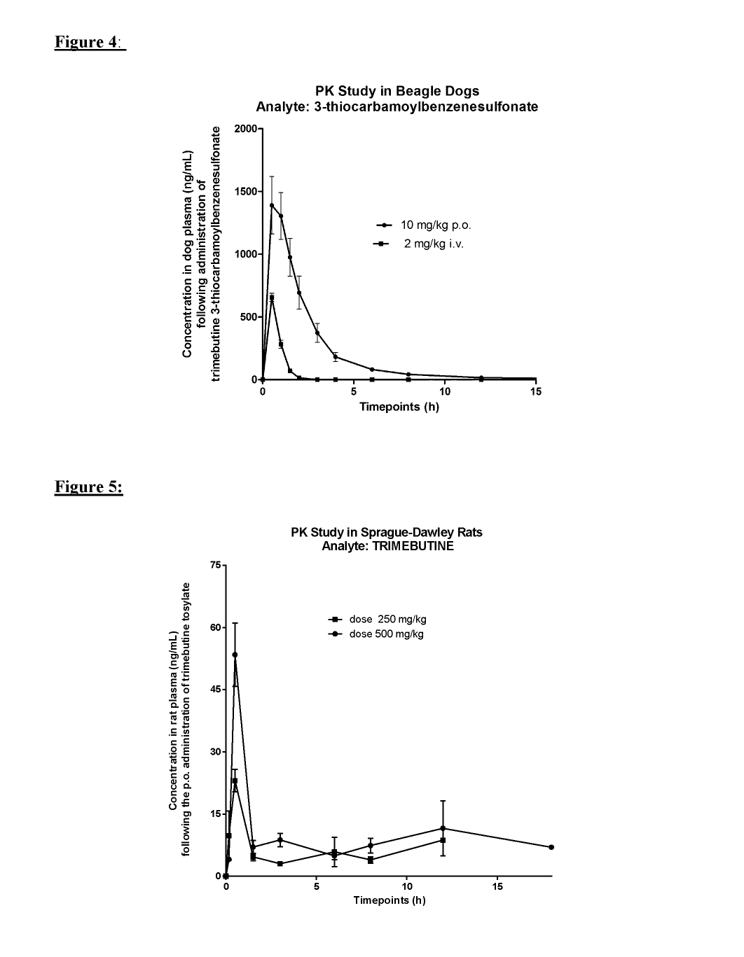

17. The compound of claim 15, wherein the polymorph has an X-ray powder diffraction pattern substantially as shown in FIG. 2 as polymorph C.

18. A pharmaceutical composition comprising at least one compound as claimed in claim 1, and a pharmaceutically acceptable excipient or carrier.

19. The pharmaceutical composition of claim 18, which is orally, parentally or intrarectally administrable in patients.

20. A method for reducing visceral pain of a patient, comprising the administration of a visceral pain relieving amount of at least one compound as defined in claim 1, to a patient in need thereof.

21. The method as claimed in claim 20, wherein the patient is undergoing lower gastrointestinal endoscopy.

22. The method as claimed in claim 20, wherein the visceral pain is due to gastrointestinal-related diseases.

23. The method as claimed in claim 20, where the patient is undergoing virtual colonoscopy or barium enema.

24. The method as claimed in claim 20, wherein the patient is undergoing upper gastrointestinal endoscopy.

25. The method of claim 22, wherein the gastrointestinal-related diseases is ulcerative colitis, internal and/or external hemorrhoids, radiation proctitis, all forms of irritable bowel syndrome or other functional disturbances of gastrointestinal motility.

26-37. (canceled)
